SEQUENCE LISTING (11 sequences)

SEQ ID NO: 1 - DNA, 2779 bp, Human
atggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctgaaacagggaaatggcacaatctgaagagacgtggtatatgatcttaaatgatg

SEQ ID NO: 2 - protein, 2779 aa, Human
ATGGTATGCGAGGGAAAGCGATCAGCCTCTTGCCCTTGTTTCTTCCTCTTGACCGCCAAGTTCTACTGGATCCTCACAATGATGCAAAGAACTCACAGCCAGGAGTATGCCCATTCCATACGGGTGGATGGGGACATTATTTTGGGGGGTCTCTTCCCTGTCCACGCAAAGGGAGAGAGAGGGGTGCCTTGTGGGGAGCTGAAGAAGGAAAAGGGGATTCACAGACTGGAGGCCATGCTTTATGCAATTGACCAGATTAACAAGGACCCTGATCTCCTTTCCAACATCACTCTGGGTGTCCGCATCCTCGACACGTGCTCTAGGGACACCTATGCTTTGGAGCAGTCTCTAACATTCGTGCAGGCATTAATAGAGAAAGATGCTTCGGATGTGAAGTGTGCTAATGGAGATCCACCCATTTTCACCAAGCCCGACAAGATTTCTGGCGTCATAGGTGCTGCAGCAAGCTCCGTGTCCATCATGGTTGCTAACATTTTAAGACTTTTTAAGATACCTCAAATCAGCTATGCATCCACAGCCCCAGAGCTAAGTGATAACACCAGGTATGACTTTTTCTCTCGAGTGGTTCCGCCTGACTCCTACCAAGCCCAAGCCATGGTGGACATCGTGACAGCACTGGGATGGAATTATGTTTCGACACTGGCTTCTGAGGGGAACTATGGTGAGAGCGGTGTGGAGGCCTTCACCCAGATCTCGAGGGAGATTGGTGGTGTTTGCATTGCTCAGTCACAGAAAATCCCACGTGAACCAAGACCTGGAGAATTTGAAAAAATTATCAAACGCCTGCTAGAAACACCTAATGCTCGAGCAGTGATTATGTTTGCCAATGAGGATGACATCAGGAGGATATTGGAAGCAGCAAAAAAACTAAACCAAAGTGGGCATTTTCTCTGGATTGGCTCAGATAGTTGGGGATCCAAAATAGCACCTGTCTATCAGCAAGAGGAGATTGCAGAAGGGGCTGTGACAATTTTGCCCAAACGAGCATCAATTGATGGATTTGATCGATACTTTAGAAGCCGAACTCTTGCCAATAATCGAAGAAATGTGTGGTTTGCAGAATTCTGGGAGGAGAATTTTGGCTGCAAGTTAGGATCACATGGGAAAAGGAACAGTCATATAAAGAAATGCACAGGGCTGGAGCGAATTGCTCGGGATTCATCTTATGAACAGGAAGGAAAGGTCCAATTTGTAATTGATGCTGTATATTCCATGGCTTACGCCCTGCACAATATGCACAAAGATCTCTGCCCTGGATACATTGGCCTTTGTCCACGAATGAGTACCATTGATGGGAAAGAGCTACTTGGTTATATTCGGGCTGTAAATTTTAATGGCAGTGCTGGCACTCCTGTCACTTTTAATGAAAACGGAGATGCTCCTGGACGTTATGATATCTTCCAGTATCAAATAACCAACAAAAGCACAGAGTACAAAGTCATCGGCCACTGGACCAATCAGCTTCATCTAAAAGTGGAAGACATGCAGTGGGCTCATAGAGAACATACTCACCCGGCGTCTGTCTGCAGCCTGCCGTGTAAGCCAGGGGAGAGGAAGAAAACGGTGAAAGGGGTCCCTTGCTGCTGGCACTGTGAACGCTGTGAAGGTTACAACTACCAGGTGGATGAGCTGTCCTGTGAACTTTGCCCTCTGGATCAGAGACCCAACATGAACCGCACAGGCTGCCAGCTTATCCCCATCATCAAATTGGAGTGGCATTCTCCCTGGGCTGTGGTGCCTGTGTTTGTTGCAATATTGGGAATCATCGCCACCACCTTTGTGATCGTGACCTTTGTCCGCTATAATGACACACCTATCGTGAGGGCTTCAGGACGCGAACTTAGTTACGTGCTCCTAACGGGGATTTTTCTCTGTTATTCAATCACGTTTTTAATGATTGCAGCACCAGATACAATCATATGCTCCTTCCGACGGGTCTTCCTAGGACTTGGCATGTGTTTCAGCTATGCAGCCCTTCTGACCAAAACAAACCGTATCCACCGAATATTTGAGCAGGGGAAGAAATCTGTCACAGCGCCCAAGTTCATTAGTCCAGCATCTCAGCTGGTGATCACCTTCAGCCTCATCTCCGTCCAGCTCCTTGGAGTGTTTGTCTGGTTTGTTGTGGATCCCCCCCACATCATCATTGACTATGGAGAGCAGCGGACACTAGATCCAGAGAAGGCCAGGGGAGTGCTCAAGTGTGACATTTCTGATCTCTCACTCATTTGTTCACTTGGATACAGTATCCTCTTGATGGTCACTTGTACTGTTTATGCCATTAAAACGAGAGGTGTCCCAGAGACTTTCAATGAAGCCAAACCTATTGGATTTACCATGTATACCACCTGCATCATTTGGTTAGCTTTCATCCCCATCTTTTTTGGTACAGCCCAGTCAGCAGAAAAGATGTACATCCAGACAACAACACTTACTGTCTCCATGAGTTTAAGTGCTTCAGTATCTCTGGGCATGCTCTATATGCCCAAGGTTTATATTATAATTTTTCATCCAGAACAGAATGTTCAAAAACGCAAGAGGAGCTTCAAGGCTGTGGTGACAGCTGCCACCATGCAAAGCAAACTGATCCAAAAAGGAAATGACAGACCAAATGGCGAGGTGAAAAGTGAACTCTGTGAGAGTCTTGAAACCAACACTTCCTCTACCAAGACAACATATATCAGTTACAGCAATCATTCAATCTGAAACAGGGAAATGGCACAATCTGAAGAGATGTGGTATATGATCTTAAATGATG

SEQ ID NO: 3 - protein, 908 aa, Human
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 4 - DNA, 2727 bp, Human
atggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaactatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctga

SEQ ID NO: 5 - protein, 908 aa, Human
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKTIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 6 - DNA, 2727 bp, Human
atggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaatactgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctga

SEQ ID NO: 7 - protein, 908 aa, Human [sequence omitted]

SEQ ID NO: 8 - DNA, 2727 bp, Human
atggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacgcggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctga

SEQ ID NO: 9 - protein, 908 aa, Human
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHAASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 10 - DNA, 2526 bp, Human
atggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacaggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctga

SEQ ID NO: 11 - protein, 841 aa, Human
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

(19) United States
(12) Patent Application Publication
(10) Pub. No.: US 2005/0233321 A1
(43) Pub. Date: Oct. 20, 2005

(54) Identification of novel polymorphic sites in the human mglur8 gene and uses thereof

(76) Inventors: John W. Hess, Lansdale, PA (US); Lee Evan Warren, Lansdale, PA (US); Peter Jeffrey Conn, North Wales, PA (US)

Correspondence Address: MERCK AND CO., INC, P O BOX 2000, RAHWAY, NJ, 07065-0907, US

(21) Appl. No.: 10/499,580

(22) PCT Filed: Dec. 19, 2002
(86) PCT No.: PCT/US02/41294

§ 371 Date: Jun. 21, 2004

Related U.S. Application Data
(60) Provisional application No. 60/342,555, filed on Dec. 20, 2001.

(51) Int. Cl. C12Q 1/68; A01K 67/00; C07H 21/04; C07K 14/705

(52) U.S. Cl. 435/6; 435/69.1; 435/320.1; 435/325; 530/350; 536/23.5; 800/8

(57) ABSTRACT

This invention relates to polymorphisms in the human mGluR8, in particular to the discovery of 10 single nucleotide polymorphisms in the mGluR8 gene. The invention also relates to methods and materials for analyzing allelic variation in the mGluR8 gene, and to the use of mGluR8 polymorphism in the diagnosis and treatment of mGluR8 and/or mGluR8-mediated diseases, such as Parkinson s disease etc. The herein disclosed probes containing at least one of the herein disclosed SNPs can be used to identify nucleic acid samples containing mGluR8 SNPs or as primers or for expressing variant proteins. Methods of analyzing the polymorphic forms occupying the polymorphic sites are also disclosed.

### CROSS-REFERENCE TO RELATED APPLICATIONS

This application claims the benefit of U.S. Provisional Application No. 60/342,555, filed Dec. 20, 2001, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

This invention relates to polymorphisms in the human metabotropic glutamate receptor subtype (mGluR8) gene. Methods and materials for analyzing allelic variation in the mGluR8 gene, and to the use of mGluR8 polymorphism in the diagnosis and treatment of mGluR8-mediated diseases, in which modulation of the mGluR8 activity could be of therapeutic benefit are also provided. Also provided are methods for detecting an individuals genetic predisposition for a disease, condition or disorder based on the presence or absence of single nucleotide polymorphisms (SNPs). Products and kits, such as panels of single nucleotide polymorphism allele specific oligonucleotides, reduced complexity genomes, and databases for diagnosing and prognosticating mGluR8-mediated or related disease by detecting a single nucleotide polymorphism in the mGluR8 gene are also provided.

### BACKGROUND OF THE INVENTION

DNA varies significantly from individual to individual, except in identical siblings. Many human diseases arise from genomic variations. The genomes of viruses, bacteria, plants and animals naturally undergo spontaneous mutation in the course of their continuing evolution (Gusella, J. F., Ann. Rev. Biochem. 55:831-854 (1986)). The genetic diversity amongst humans and other life forms explains the heritable variations observed in disease susceptibility. It has been estimated that variations in genomic DNA sequences are created continuously at a rate of about 100 new single base changes per individual (Kondrashow, L T. Theor. Biol., 175:583-594, 1995; Crow, Exp. Clin. Immunogenet., 12:121-128, 1995). Over time, a significant number of mutations can accumulate within a population such that considerable polymorphism can exist between individuals within the population.

Several different types of polymorphism have been reported which result in genetic diversity. A restriction fragment length polymorphism (RFLP) means a variation in DNA sequence that alters the length of a restriction fragment as described in Botstein et al., Am. J. Hum. Genet. 32, 314-331 (1980). The restriction fragment length polymorphism may create or delete a restriction site, thus changing the length of the restriction fragment. RFLPs have been widely used in human and animal genetic analyses (see WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the animal will also exhibit the trait.

Variable number of tandem repeats (“VNTRs”), arise from spontaneous tandem duplications of di- or trinucleotide repeated motifs of nucleotides (Weber, J. L., U.S. Pat. No. 5,075,217; Armour, J. A. L. et al., FEBS Lett. 307:113-115 (1992); Jones, L. et al., Eur. J. Haematol. 39:144-147 (1987); Horn, G. T. et al., PCT Application WO91/14003; Jeffreys, A. J., European Patent Application 370,719; Jeffreys, A. J., U.S. Pat. No. 5,175,082); Jeffreys, A. J. et al., Amer. J. Hum. Genet. 39:11-24 (1986); Jeffreys, A. J. et al., Nature 316:76-79 (1985); Gray, I. C. et al., Proc. R. Acad. Soc. Lond. 243:241-253 (1991); Moore, S. S. et al., Genomics 10:654-660 (1991); Jeffreys, A. J. et al., Anim. Genet. 18:1-15 (1987); Hillel, J. et al., Anim. Genet. 20:145-155 (1989); Hillel, J. et al., Genet. 124:783-789 (1990)). VNTRs have been used in identity and paternity analysis (U.S. Pat. No. 5,075,217; Armour et al., FEBS Lett. 307, 113-115 (1992); Horn et al., WO 91/14003; Jeffreys, EP 370,719), and in a large number of genetic mapping studies.

By far the most common source of variation in the genome are “single nucleotide polymorphisms” or SNPs, which account for approximately 90% of human DNA polymorphism (Collins et al., Genome Res., 8:1229-1231, 1998). SNPs are single base pair positions in genomic DNA at which different sequence alternatives (alleles) exist in a population. Several definitions of SNPs exist in the literature (Brooks, Gene, 234:177-186, 1999). A central attribute of such a polymorphism is that it contains a polymorphic site, “X,” most preferably occupied by a single nucleotide, which is the site of the polymorphism's variation (Goelet, P. and Knapp, M., U.S. patent application Ser. No. 08/145,145, herein incorporated by reference).

SNPs can arise in several ways. A SNP may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or the converse.

SNPs can also arise from a deletion of a nucleotide or an insertion of a nucleotide relative to a reference allele. Thus, the polymorphic site is a site at which one allele bears a gap with respect to a single nucleotide in another allele. Some SNPs occur within, or near genes.

SNPs can be associated with disease conditions in humans or animals. One such class includes SNPs falling within regions of genes encoding for a polypeptide product. These SNPs may result in an alteration of the amino acid sequence of the polypeptide product and give rise to the expression of a defective or other variant protein. Such variant products can, in some cases result in a pathological condition, e.g., genetic disease. Examples of genes in which a polymorphism within a coding sequence gives rise to genetic disease include sickle cell anemia and cystic fibrosis.

More frequently, SNPs occur in non coding regions and as such do not result in alteration of the polypeptide product. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

The association between a SNP and a disease state can also be indirect where the SNP does not directly cause the disease but alters the physiological environment such that there is an increased likelihood that the patient will develop the disease.

Lastly, SNPs can also be associated with disease conditions, but play no direct or indirect role in causing the disease. In this case, the SNP is located close to the defective gene such that there is a strong association between the presence of the SNP and the disease state. Because of the high frequency of SNPs within the genome, there is a greater probability that a SNP will be linked to a genetic locus of interest than other types of genetic markers.

There are numerous reasons why SNPs are especially suited for the identification of genotypes that influence an individual's predisposition to a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers, but as candidates for gene therapy treatments to treat or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations. Fifth, the greater frequency of SNPs means that they can be more readily identified than the other classes of polymorphisms. Sixth, the greater uniformity of their distribution permits the identification of SNPs “nearer” to a particular trait of interest. The combined effect of the above referenced attributes makes SNPs extremely valuable. For example, if a particular trait (e.g. predisposition to cancer) reflects a mutation at a particular locus, then any polymorphism that is linked to the particular locus can be used to predict the probability that an individual will be exhibit that trait. Also because SNPs result from sequence variation, new polymorphisms can be identified by sequencing random genomic or cDNA molecules.

In summary, DNA polymorphisms may lead to variations in amino acid sequence and consequently to altered protein structure and functional activity. Polymorphisms may also affect mRNA synthesis, maturation, transportation and stability. Polymorphisms which do not result in amino acid changes (silent polymorphisms) or which do not alter any known consensus sequences may nevertheless have a biological effect, for example by altering mRNA folding or stability.

Numerous methods exist for the detection of SNPs within a nucleotide sequence. A review of many of these methods can be found in Landegren et al., Genome Res., 8:769-776, 1998. Methods for the detection of specific mutations include allele specific primer extension, allele specific probe ligation, and differential probe hybridization. See, for example, U.S. Pat. Nos. 5,888,819; 6,004,744; 5,882,867; 5,710,028; 6,027,889; 6,004,745; and WO US88/02746.

Various microsequencing methods for assaying polymorphic sites in DNA have also been described. See Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoll, L. et al., GATA 9:107-112 (1992); Nyren, P. et al., Anal. Biochem. 208:171-175 (1993)).

The detection of sequence alterations in a nucleic acid sequence is important for the detection of mutant genotypes, as relevant for genetic analysis, the detection of mutations leading to drug resistance, pharmacogenomics, etc. Although many of the variations in the genome do not result in a disease trait, some do. Diseases arising from single gene mutation include Huntington's disease, cystic fibrosis, Duchenne muscular dystrophy, and certain forms of breast cancer. Diseases such as multiple sclerosis, diabetes, Parkinson's, Alzheimer's disease, and hypertension are much more complex. These diseases may be due to polygenic (multiple gene influences) or multifactorial (multiple gene and environmental influences) causes.

Anatomical, biochemical and electrophysiological analyses suggest that glutamatergic systems are involved in a broad array of neuronal processes, including fast excitatory synaptic transmission, regulation of neurotransmitter releases, long-term potentiation, learning and memory, developmental synaptic plasticity, hypoxic-ischemic damage and neuronal cell death, epileptiform seizures, as well as the pathogenesis of several neurodegenerative disorders. See generally, Monaghan et al., Ann. Rev. Pharmacol. Toxicol. 29:365-402 (1980). This extensive repertoire of functions, especially those related to learning, neurotoxicity and neuropathology, has stimulated recent attempts to describe and define the mechanisms through which glutamate exerts its effects. See Watkins & Evans, Annual Reviews in Pharmacology and Toxicology, 21:165 (1981); Monaghan, Bridges, and Cotman, Annual Reviews in Pharmacology and Toxicology, 29:365 (1989); Watkins, Krogsgaard-Larsen, and Honore, Transactions in Pharmaceutical Science, 11:25 (1990).

Current methods for identifying pharmaceuticals to treat disease often start by identifying, cloning, and expressing an important target protein related to the disease. A determination of whether an agonist or antagonist is needed to produce an effect that may benefit a patient with the disease is then made. Then, vast numbers of compounds are screened against the target protein to find new potential drugs. The desired outcome of this process is a drug that is specific for the target, thereby reducing the incidence of the undesired side effects usually caused by a compound's activity at non-intended targets. A chief drawback attending this approach is that it fails to consider that natural variability exists in any and every population with respect to a particular protein. A target protein currently used to screen drugs typically is expressed by a gene cloned from an individual who was arbitrarily selected. However, the nucleotide sequence of a particular gene may vary tremendously among individuals. Subtle alteration(s) in the primary nucleotide sequence of a gene encoding a target protein may be manifested as significant variation in expression of or in the structure and/or function of the protein. Such alterations may explain the relatively high degree of uncertainty inherent in treatment of individuals with drugs whose design is based upon a single representative example of the target.

For example, it is well-established that some classes of drugs frequently have lower efficacy in some individuals than others, which means such individuals and their physicians must weigh the possible benefit of a larger dosage against a greater risk of side effects. Accordingly, SNPs associated with a particular disease status or a gene may aid in the design of a more efficacious treatment protocol as well as identification of a better suited therapeutic product. In this regards the herein identified SNPs relating to the human mGluR8 gene will be very useful.

Polymorphisms may also be used in mapping the human genome and to elucidate the genetic component of diseases. The reader is directed to the following references for background details on pharmacogenetics and other uses of polymorphism detection: Linder et al., (1997), Clinical Chemistry, 43, 254; Marshall (1997), Nature Biotechnology, 15, 1249; International Patent Application WO 97/40462, Spectra Biomedical; and Schafer et al., (1998), Nature Biotechnology, 16, 33.

It is well recognized that the ability to scan the human genome to identify the location of genes which underlie or are associated with the pathology of such diseases is an enormously powerful tool in medicine and human biology. In fact, it is widely recognized that SNPs can provide a powerful tool for studying sequence variations in individuals whose genetic make-up alters their susceptibility to certain diseases. As such, the ability to screen an individual for a specific polymorphism which may underlie or be associated with the pathology of a disease state will is an enormously powerful tool in medicine and human biology. The identification of an individual's genetic profile can require the identification of particular nucleic acid sequences in the individual's genome. These particular nucleic acid sequences can include those that differ by one or a few nucleotides among individuals in the same species.

Pathologies associated with defects in the modulation of the human mGluR8 receptor subtype conform to a broad clinical spectrum. The paucity of disease states involving the mGluR8 receptor include but are not limited to schizophrenia, Parkinson's disease, Alzheimer's disease, Huntington's disease, stroke, anxiety, cognitive dysfunction, attention deficit hyperactivity disorder, autism, pain and inflammation.

As such, knowledge of polymorphisms of the human mGluR8 gene may be used to help identify patients most suited to therapy with particular pharmaceutical agents (this is often termed “pharmacogenetics”). Pharmacogenetics can also be used in pharmaceutical research to assist the drug selection process.

The mGluR8 receptor is one of at least many glutamate receptors in the body. In general, pharmacotherapeutic compounds used to treat many diseases work by activating a receptor or inhibiting the action of its natural ligand.

Several recent reports have suggested a role for human mGluR8 encoding gene in the pathogenesis of Schizophrenia. See Bolonna et al., Schizophr. Res., 47: 99-103 (2001). See also Harrison, et al., Lancet 337: 450-452, who report on decreased hioppocampal expression of a glutamate receptor gene linked to Schizophrenia. (1991).

Several polymorphic regions that are associated with specific diseases or disorders, have been linked in the human to the mGluR8 gene by analyzing the DNA of a specific population of individuals. Variations in the human mGluR8 receptor amongst the population are known to be caused by allelic variation, and this variation can alter the response of a disease to a drug amongst patients. One polymorphism (variation) found in the population is a change from a isoleucine (Ile) to a threonine (Thr) at position 256 (I256T) of the mGluR8 receptor. A second polymorphism is a change from a cytosine to thymine (C2846T).

Genetic screening (also called genotyping or molecular screening), can be broadly defined as testing to determine if a patient has mutations (alleles or polymorphisms) that either cause a disease state or are “linked” to the mutation causing a disease state. Linkage refers to the phenomenon where DNA sequences which are close together in the genome have a tendency to be inherited together.

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, mGluR8 mediated disease states. With the development of simple and inexpensive genetic screening methodology, it is now possible to identify polymorphisms that indicate a propensity to develop disease, even when the disease is of polygenic origin.

Because of the potential for polymorphisms in the human mGluR8 gene to affect the expression and function of the encoded protein, it would be useful to determine whether additional polymorphisms exist in the human mGluR8 gene, as well as how such polymorphisms are combined in different copies of the gene. Such information would be useful for studying the biological function of human mGluR8 as well as in identifying drugs targeting this protein for the treatment of disorders related to its abnormal expression or function. Nucleic acid analysis of the human mGluR8 gene will aid in the identification of defined phenotypes, diagnosis of genetic diseases as well as to the susceptibility to a disease, assessment of gene expression in development, disease and in response to defined stimuli, as well as the various genome projects. Knowledge of mGluR8 SNPs may be used to effectively delay, or, ideally, prevent onset of such disease states.

To reiterate, the polymorphisms identified herein as they relate to the human mGluR8 receptor subtype gene will aid in the diagnosis and prognosis of individuals susceptible to such conditions base upon the presence or absence of a specific SNP. The medical consequences of such SNP makes the abatement of the aforementioned disease states attending mGluR8 SNPs an important therapeutic goal.

### SUMMARY OF THE INVENTION

The invention is based, in part, on the discovery and identification of polymorphic regions within the gene encoding for human mGluR8, which has previously been associated with specific diseases or disorders, including Schizophrenia. In particular, the inventors have discovered novel single nucleotide polymorphisms (SNP) in regions of human mGluR8 subtype gene, which has been mapped to chromosome 7q31.3-q32.1. The structure of the gene (reference gene) is detailed on SEQ ID NO:1.

Specifically, the inventors herein have discovered 10 novel polymorphic sites in the human mGluR8 gene. Of these, 5 are located in the coding region, and 5 are located in the untranslated region, i.e., introns etc. The polymorphic sites of the coding regions (PS) correspond to the following nucleotide positions in SEQ ID NO:1¹: 1,392,239 which represents a thymine for cytosine (TTC>TTT) (PS1¹); 1,528,555 which represents cytosine for thymine (GGT>GGC) (PS2); 1,730,468 which represents cytosine for thymine (ATT>ACT) in the coding region of the receptor (PS3); 1,730,897 which represents a guanine for adenine (TAA>TAG) (PS4), 1,731,127 which represents guanine for adenine (CCA>CCG) (PS5); 1,732,472 which represents adenine for thymine (TTC>TAC) in the coding region of the receptor (PS6); 1,865,017 which represents adenine for cytosine (CAG>AAG) in the consensus splice site for exon 6 of the receptor (PS7); 2,101,189 which represents cytosine for thymine (TAT>TAC) (PS8); 2,101,237 which represents guanine for cytosine (CCC>CGC) in the coding region of the receptor (PS9); and 2,195,995 which represents cytosine for thymine (GAT>GAC) corresponding to the 3′ untranslated region of human mGluR8 mRNA transcript derived from the GRM8 gene (PS10).
¹ Each novel SNP with the upstream and downstream 50 nucleotide bases is listed in FIGS. 4(a)-(b) (PS1-10)

In addition, the inventors have determined the identity of the alternative nucleotides present at these sites in a human reference population of 50 unrelated individuals. Specifically, 10 exons of human mGluR8 were sequenced from 50 individuals that were obtained from the Coriell cell repository.

A genomic DNA sequence encoding human mGluR8 receptor subtype has been sequenced, assembled and deposited in GenBank database, accession number NT 007933. A 829,973 nucleotide sequence that contains all of the mGluR8 exons and corresponds to nucleotides 1,292,101 to 2,122,073 of the Feb. 9, 2001 was used as an initial reference sequence for primer design etc. The updated version of NT 007933 was used as a reference sequence-NT 007933.7-last updated Dec. 10, 2001 for location of the herein described SNPs. The reference sequence was utilized to position exons, design primers for amplification of exons by PCR. The position of polymorphisms within the reference sequence NT 007933.7 of 7,106,047 nucleotides updated Dec. 10, 2001 is presented in Table 1. Thus, all positions in the coding region and 3′untranslated region of the human mGluR8 receptor subtype gene herein refer to the positions in SEQ ID NO:1 (which is equivalent to EMBL accession number NT 007933.7 last modified on Dec. 10, 2001 and the version in the National Center for Biotechnology Information database at the time of filing this application) unless stated otherwise or apparent from the context.

cDNA sequence(s) encoding human mGluR8 has published and is designated XM 045464. Its nucleotide sequence corresponds to SEQ ID NO:2 herein. Other cDNA sequences encoding human mGluR8 have also published as U.S. Pat. Nos. 6,221,609, and 6,084,084.

It is believed that human mGluR8—encoding polynucleotides containing one or more of the novel polymorphic sites reported herein will be useful in studying the expression and biological function of human mGluR8, as well as in developing drugs targeting this protein. In addition, information on the combinations of polymorphisms in the human mGluR8 gene may have diagnostic and forensic applications.

Accordingly, an aspect of the invention provides a nucleic acid molecule or polynucleotide that includes one or more of the herein described SNPs. The nucleic acid molecule can be, e.g., a nucleotide sequence which includes one or more of the polymorphic sequences disclosed herein, or a fragment of the polymorphic sequence, with the proviso that it includes the polymorphic site. The nucleic acid molecule may alternatively contain a nucleotide sequence that is complementary to one or more of the above polymorphic sequences, or a fragment of the complementary nucleotide sequence, provided that the fragment includes a polymorphic site. The disclosed single nucleotide polymorphism(s) is/are generally associated with a human mGluR8 mediated disorder such as Schizophrenia, Parkinson's disease, Alzheimer's disease, Huntington's disease, stroke, anxiety, cognitive dysfunction, attention deficit hyperactivity disorder, autism, pain and inflammation.

As a consequence, an embodiment of the invention provides a nucleic acid molecule comprising a nucleotide sequence which is a polymorphic variant of a reference sequence for the human mGluR8 gene or a fragment thereof. The reference sequence comprises SEQ ID NO:1 and the polymorphic variant comprises at least one polymorphism selected from the group consisting of PS1 through PS10—that is thymine at 1,392,239 (PS1), cytosine at 1,528,555(PS2), cytosine at 1,730,468 (PS3),

Guanine at position 1,730,897 (PS4); guanine at 1,731,127 (PS5); adenine at 1,732,472 (PS6); adenine at 1,865,017(PS7), cytosine at 2,101,189 (PS8); guanine at 2,101,237(PS9); and cytosine at 2,195,995 (PS10). Refer to Table 5 which details PS1-PS10, with the corresponding change in amino acid. For ease of understanding, representative fragments (PS1-PS10) detailing each polymorphic site of the invention are shown in Table 5.

In another embodiment, the invention provides a nucleic acid molecule e comprising a polymorphic variant of a reference sequence for a human mGluR8 cDNA or a fragment thereof. The reference sequence comprises SEQ ID NO:2, wherein the polymorphic cDNA/mRNA comprises at least one SNP located at one of the positions defined herein: thymine for cytosine at a position corresponding to nucleotide position 357 and is equivalent to PS1 in the genomic sequence; cytosine for thymine at a position corresponding to nucleotide 693 (GGT>GGC) and is equivalent to PS2; cytosine at a position corresponding to nucleotide 794 resulting in an amino acid change from isoleucine to threonine (Ile265Thr) and is equivalent to PS3; adenine at a position corresponding to nucleotide 1095 resulting in a change in amino acid from phenyalaline to tyrosine (Phe362Tyr) and is equivalent to PS6, and guanine at a position corresponding to nucleotide 1534 resulting in a change in amino acid from proline to alanine (Pro512Ala) and is equivalent to PS9.

Polynucleotides complementary to these human mGluR8 genomic and cDNA variants are also provided herein.

In another embodiment, the invention provides a recombinant expression vector comprising one of the polymorphic genomic or cDNA variants operably linked to expression regulatory elements as well as a recombinant host cell transformed or transfected with the expression vector. The recombinant vector and host cell may be used to express human mGluR8 for protein structure analysis and drug binding studies.

The invention further provides allele-specific oligonucleotides that hybridize to a nucleic acid or its complement, including the polymorphic site(s). Such oligonucleotides are useful as probes or primers which can detect the polymorphisms of the invention.

According to another aspect of the present invention there is provided an allele specific primer capable of detecting a mGluR8 subtype gene polymorphism selected from the group consisting of 1,392,239; 1,528,555; 1,730,468; 1,730,897; 1,731,127; 1,732,472; 1,865,017; 2,101,189; 2,101,237 and 2,195,995 relative to SEQ ID NO: 1; or positions 357, 693, 794, 1095 and 1534 in SEQ ID NO:2 or a gene encoding a polymorphic variant of a nucleic acid molecule encoding a polypeptide variant as described infra.

An allele-specific primer is generally used together with a constant primer, in an amplification reaction such as a PCR reaction, which provides the discrimination between alleles through selective amplification of one allele at a particular sequence position e.g. as used for ARMS™ assays. The allele-specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer. Primers may be manufactured using any convenient method of synthesis.

Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1″ Edition. If required the primer(s) may be labeled to facilitate detection.

According to another aspect of the present invention there is provided an allele-specific oligonucleotide probe capable of detecting a human mGluR8 polymorphism at one or more of the positions identified herein.

The allele-specific probes are of any convenient length such as up to 50 bases, up to 40 bases, more conveniently up to 30 bases in length, such as for 17-50 nucleotides, more preferably about 17-35 nucleotides, and more preferably about 17-30 nucleotides. The design of such probes will be apparent to the molecular biologist of ordinary skill. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection. Such labels are well known to a skilled artisan.

The methods involve identifying a nucleotide or nucleotide pair present at one or more of the polymorphic sites detailed herein in one or both copies of the human mGluR8 gene from an individual. Alternatively, identifying a nucleotide at one or more polymorphic sites corresponding to nucleotides 357, 693, 794, 1095 and 1534 in an mRNA sample relative to XM-045464, SEQ ID NO:2. The method includes contacting the nucleic acid with an oligonucleotide probe that hybridizes to a polymorphic sequence containing at least one of the SNPs detailed herein or its complement.

The method also includes determining whether the nucleic acid and the oligonucleotide probe hybridize. Hybridization of the oligonucleotide to the nucleic acid sequence indicates the presence of the polymorphic site in the nucleic acid. The oligonucleotide probes can vary in lengths as discussed supra.

In another aspect, the invention provides an oligonucleotide array comprising one or more oligonucleotide probes hybridizing to a first polynucleotide at a polymorphic site encompassed therein. The first polynucleotide can be, e.g., a nucleotide sequence comprising one or more polymorphic sequences defined herein, a nucleotide sequence that is a fragment of any of the polymorphic nucleotide sequence disclosed herein, provided that the fragment includes a polymorphic site in the polymorphic sequence; a complementary nucleotide sequence comprising a sequence complementary to one or more polymorphic sequences of the invention; or a nucleotide sequence that is a fragment of the complementary sequence, provided that the fragment includes a polymorphic site in the polymorphic sequence. In preferred embodiments, the array comprises 10; 100; 1,000; 10,000; 100,000 or more oligonucleotides.

A polymorphic variant of human mGluR8 polypeptide is useful in studying the effect of the variation on the biological activity of human mGluR8 as well as studying the binding affinity of candidate drugs targeting human mGluR8 for the treatment of human mGluR8 mediated disorders.

Consequently, an embodiment of the invention provides a polypeptide comprising a polymorphic variant of a reference amino acid sequence for the human mGluR8 receptor protein. The reference amino acid sequence comprises SEQ ID NO:3 and the polymorphic variant comprises Threonine at a position corresponding to amino acid position 265, Tyrosine at a position corresponding to amino acid position 362, and Alanine at a position corresponding to amino acid position 512 as shown in the reference sequence, or a fragment thereof comprising at least one of the aforementioned variants therein. Polynucleotides encoding the variant polypeptide are also within the scope of the invention as are probes and primers for detecting these nucleic acid molecules.

The present invention also provides antibodies that recognize and bind to the polymorphic variant polypeptide referenced supra. Such antibodies can be utilized in a variety of diagnostic and prognostic formats and therapeutic methods.

The present invention also provides transgenic animals comprising one of the human mGluR8 polymorphic variant nucleic acid molecules described herein and methods for producing such animals. The transgenic animals are useful for studying expression of the human mGluR8 in vivo, for in vivo screening and testing of drugs targeted against the human mGluR8 protein, and for testing the efficacy of therapeutic agents and compounds for human mGluR8 mediated disorders in a biological system.

The methods of the invention are also useful for detecting variants of a nucleic acid sequence contained in a target nucleic acid for example in detecting SNPs in a nucleic acid sequence of interest (e.g., alleles) and, optionally, to identifying such SNPs or alleles.

Another aspect of the invention provides a method for the diagnosis of a SNP in a human mGluR8 subtype gene in a human, which method comprises determining the sequence of the gene obtained from the human; and determining the status of the human by reference to polymorphism in the human mGluR8 subtype gene.

In one embodiment of the invention preferably the method for diagnosis described herein is one in which the SNP at position 1,392,239 is presence of thymine (T) for cytosine (C) in SEQ ID NO: 1 (the published base)—PS1.

In another embodiment of the invention preferably the method for diagnosis described herein is one in which the SNP at position 1,528,555 is presence of cytosine for thymine (C for T), relative to the published base—PS2.

Allelic variation at position 1,730,468 consists of a single base substitution from thymine (T)(the published base), preferably to cytosine (C)—PS3.

Allelic variation at position 1,730,897 consists of a single base substitution from adenine (A) (the published base), preferably to guanine (G)—PS4.

Allelic variation at position 1,731,127 consists of a single base substitution from adenine (A) (the published base), preferably to guanine (G)—PS5.

Likewise, allelic variation at position 1,732,472 consists of a single base substitution from thymine (T) (the published base), preferably to adenine (A)—PS6.

Allelic variation at position 1,865,017 consists of a single base substitution from cytosine (C) (the published base), to adenine (A)—PS7.

Allelic variation at position 2,101,189 consists of a single base substitution from thymine (T) (the published base), to cytosine (C)—PS8.

Allelic variation at position 2,101,237 consists of a single base substitution from cytosine (C) (the published base), to guanine (G)—PS9.

Allelic variation at position 2,195,995 consists of a single base substitution from thymine (T) (the published base), to cytosine (C)—PS10.

The status of the individual may be determined by reference to allelic variation at any one or more positions optionally in combination with any other polymorphism in the gene that is (or becomes) known. The test sample of nucleic acid is conveniently a sample of blood, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g., PCR, before analysis of allelic variation. It will be apparent to the person skilled in the art that there are a large number of analytical procedures, which may be used to detect the presence or absence of variant nucleotides at one or more polymorphic positions of the invention.

In a further diagnostic aspect of the invention the presence or absence of variant nucleotides is detected by reference to the loss or gain of, optionally engineered, sites recognized by restriction enzymes.

In a further aspect, the diagnostic methods of the invention are used to assess the efficacy of therapeutic compounds in the treatment of a human mGluR8 mediated disease such as those in which perturbation of the glutametergic system may participate. These include but are not limited to Schizophrenia, Parkinson's disease, Alzheimer's disease, Huntington's disease, stroke, anxiety, cognitive dysfunction, attention deficit hyperactivity disorder, autism, pain and inflammation.

Assays, for example reporter-based assays, may be devised to detect whether one or more of the above polymorphisms affect transcription levels and/or message stability. Individuals who carry particular allelic variants of the human mGluR8 subtype gene such as those identified herein may therefore exhibit differences in their ability to regulate protein biosynthesis resulting from modulation of the mGluR8 gene or its gene product under different physiological conditions and may display altered abilities to react to different diseases.

In addition, differences in receptor modulation and its attending second messenger activity such as protein regulation or target gene transcription arising as a result of allelic variation may have a direct effect on the response of an individual to drug therapy.

The diagnostic methods of the invention may be useful both to predict the clinical response to such agents and to determine therapeutic dose.

Polymorphisms are also useful in mapping the human genome and to elucidate the genetic component of diseases. As such, the herein-disclosed SNPs will provide method(s) for diagnosing a genetic predisposition for the development of a mGluR8-mediated disease in an individual(s). Information obtained from the detection of SNPs associated with an individual's genetic predisposition to a disease is of great value in the treatment and prevention of the disease. As such, the herein-disclosed SNPs may also be used to recognize individuals who are particularly at risk from developing these conditions.

Accordingly, an aspect of the present invention provides a method for diagnosing a genetic predisposition for a disease, condition or disorder in a subject comprising, obtaining a biological sample containing nucleic acid from said subject; and analyzing the nucleic acid to detect the presence or absence of any one of the herein disclosed SNPs relative to SEQ ID NO: 1 or the complement thereof, wherein the SNP is associated with a genetic predisposition for a disease condition or disorder selected from the group consisting of schizophrenia, Parkinson's disease, Alzheimer's disease, Huntington's disease, stroke, anxiety, cognitive dysfunction, attention deficit hyperactivity disorder, autism, pain and inflammation.

Accordingly, one aspect of the present invention provides a method for diagnosing a genetic predisposition for a disease, condition or disorder in a subject comprising, obtaining a biological sample containing nucleic acid from said subject; and analyzing said nucleic acid to detect the presence or absence of any one or more of the SNP disclosed herein relative to SEQ ID NO: 1 or SEQ ID NO:2 or SEQ ID NO:3 or the complement thereof, wherein said SNP is associated with a genetic predisposition for a human mGluR8 mediated disease condition or disorder. This may be particularly relevant in the development of Schizophrenia, Parkinson's disease, Alzheimer's disease, Huntington's disease, stroke, anxiety, cognitive dysfunction, attention deficit hyperactivity disorder, autism, pain and inflammation diseases and other diseases which are modulated by human mGluR8 receptor interactions.

In another aspect, the method entails determining the sequence of nucleotide of a gene obtained from a subject and determining the nucleotides at positions 1,392,239; 1,528,555; 1,730,468; 1,730,897; 1,731,127; 1,732,472; 1,865,017; 2,101,189; 2,101,237 and 2,195,995 and correlating the frequency of occurrence of said polymorphism in a population as well as the frequency of said polymorphism as it relates to a specific disease condition associated with said polymorphism.

Alternatively, the method comprises determining the amino acid sequence of the gene product of the gene isolated from said subject and determining the amino acids at positions 265, 362 and 512 relative to SEQ ID NO:3 and correlating the frequency of occurrence of any one of the herein disclosed polymorphisms at one or more of the above positions in a population as well as the frequency of said polymorphism as it relates to a specific disease condition associated with said polymorphism.

The nucleic acids can be DNA or RNA. Some nucleic acids contain a polymorphic site having two polymorphic forms giving rise two different amino acids specified by the two codons in which the polymorphic site occurs in the two polymorphic forms.

In a further aspect, the diagnostic methods of the invention are used in the development of new drug therapies, which selectively target one or more allelic variants of the human mGluR8 subtype gene. Identification of a link between a particular allelic variant and predisposition to disease development or response to drug therapy may have a significant impact on the design of new drugs. Drugs may be designed to regulate the biological activity of variants implicated in the disease process whilst minimizing effects on other variants.

In a further diagnostic aspect of the invention the presence or absence of variant nucleotides is detected by reference to the loss or gain of, optionally engineered, sites recognized by restriction enzymes.

The novel sequence that include at least one of the polymorphisms disclosed herein, may be used in another embodiment of the invention to regulate expression of the gene in cells by the use of antisense constructs. To enable methods of down-regulating expression of the polymorphic gene of the present invention in mammalian cells, an example antisense expression construct can be readily constructed for instance using the pREP10 vector (Invitrogen Corporation). Transcripts are expected to inhibit translation of the polymorphic gene(s) in cells transfected with this type construct. Antisense transcripts are effective for inhibiting translation of the native gene transcript, and capable of inducing the effects (e.g., regulation of tissue physiology) herein described.

The invention also provides a method of treating a subject suffering from, at risk for, or suspected of, suffering from pathology ascribed to the presence of a sequence polymorphism in a subject, e.g., a human, non-human primate, cat, dog, rat, mouse, cow, pig, goat, or rabbit. The polymorphic site preferably encompasses at least one or a combination of the herein-disclosed SNPs.

According to another aspect of the present invention there is provided a method of treating a human in need of treatment with a human mGluR8 receptor antagonist drug in which the method comprises: i) diagnosis of a SNP in the mGluR8 gene in the human, which diagnosis comprises determining the sequence of the nucleic acid at one or more polymorphic sites identified herein; and determining the status of the human by reference to polymorphism in the mGluR8 subtype gene; and ii) administering an effective amount of a human mGluR8 receptor antagonist.

The mGluR8 receptor antagonist drug may act directly on the receptor and/or its binding partner. These have been reviewed in P. Jeffrey Conn and Jean-Philippe Pin Pharmacology and Functions of Metabotropic Glutamate Receptors Annu. Rev. Pharmacol. Toxicol. 1997. 37:205-237.

According to another aspect of the present invention there is provided a pharmaceutical pack comprising a human mGluR8 receptor antagonist drug and instructions for administration of the drug to humans diagnostically tested for a SNP at one or more of positions allelic variations (SNPs) disclosed herein.

In other embodiments, the invention provides methods, compositions, and kits for haplotyping and/or genotyping the human mGluR8 gene in an individual.

The methods and compositions for establishing the genotype or haplotype of an individual at the novel polymorphic sites described herein are useful for studying the effect of the polymorphisms in the etiology of diseases affected by the expression and function of the human mGluR8 protein, studying the efficacy of drugs targeting human mGluR8, predicting individual susceptibility to diseases affected by the expression and function of the human mGluR8 protein and predicting individual responsiveness to drugs targeting human mGluR8. The compositions contain oligonucleotide probes and primers designed to specifically hybridize to one or more target regions containing, or that are adjacent to, a polymorphic site.

A haplotype is a set of alleles found at linked polymorphic sites (such as within a gene) on a single (paternal or maternal) chromosome. If recombination within the gene is random, there may be as many as 2^{II} haplotypes, where 2 is the number of alleles at each SNP and n is the number of SNPs.

One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel SNPs in the vWF gene promoter. Blood (1999) 93:4277-83, Keightley A M, Lam Y M, Brady J N, Cameron C L, Lillicrap D).

Preferably determination of the status of the human is clinically useful. Examples of clinical usefulness include deciding which antagonist drug or drugs to administer and/or in deciding on the effective amount of the drug or drugs. Human mGluR8 subunit ligand antagonist drugs have been disclosed in the following publications: Thomas, N. K., Wright, R. A., Howson, P. A., Kingston, A. E., Schoepp, D. D., and Jane, D. A., “(S)-3,4-DCPG, a potent and selective mGluR8 receptor agonist, activates metabotropic glutamate receptors on primary afferent terminals in the neonatal rat spinal cord,” Neuropharmacology, 40:311-318 (2001).

The invention also provides methods of screening polymorphic sites linked to at least one or more polymorphic sites disclosed herein for suitability for diagnosing a phenotype. Such methods entail identifying a polymorphic site in the human mGluR8 gene linked to at least one of the polymorphic sites disclosed herein, wherein a polymorphic form of the polymorphic site disclosed herein has been correlated with a phenotype. One then determines haplotypes in a population of individuals to indicate whether the linked polymorphic site has a polymorphic form in equilibrium dislinkage with the polymorphic form correlated with the phenotype.

In yet another embodiment, the invention provides a method for identifying an association between a genotype or haplotype and a trait. In preferred embodiments, the trait is susceptibility to a disease, severity of a disease, the staging of a disease or response to a drug. Such methods have applicability in developing diagnostic tests and therapeutic treatments for cancers, inflammatory and immune disorders.

According to another aspect of the present invention there is provided a computer readable medium comprising at least one nucleotide sequence having at least one of the novel polymorphic sites therein stored on the medium. The computer readable medium may be used, for example, in homology searching, mapping, haplotyping, genotyping or pharmacogenetic analysis or any other bioinformatic analysis. The reader is referred to Bioinformatics, A practical guide to the analysis of genes and proteins, Edited by A D Baxevanis & B F F Ouellette, John Wiley & Sons, 1988. Any computer readable medium may be used, for example, compact disk, tape, floppy disk, hard drive or computer chips. The nucleotide sequences of the invention, or parts thereof, particularly those relating to and identifying the SNPs identified herein represent a valuable information source, for example, to characterize individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases using state of the art searching tools such as “GCG”.

Thus, nucleotide sequences containing at least one of the herein disclosed polymorphic sites are particularly useful as components in databases useful for sequence identity and other search analyses. As used herein, storage of the sequence information in a computer readable medium and use in sequence databases in relation to ‘polynucleotide or polynucleotide sequence of the invention’ covers any detectable chemical or physical characteristic of a polynucleotide of the invention that may be reduced to, converted into or stored in a tangible medium, such as a computer disk, preferably in a computer readable form.

Consequently, there is provided herein a computer readable medium having stored thereon one or a more nucleotide sequences having contained therein at least one of the novel polymorphisms described herein. For example, a computer readable medium is provided comprising and having stored thereon a member selected from the group consisting of a nucleotide sequence comprising at least of the herein disclosed polymorphic sites, a fragment thereof that includes a polymorphic site, and a set of sequences wherein the set includes at least one sequence containing therein at least on eof the herein disclosed polymorphisms, a data set comprising or consisting of a nucleotide comprising at least one of the polymorphisms disclosed herein or a part thereof comprising at least one of the polymorphisms identified herein.

A computer based method is also provided for performing sequence identification, the method comprising the steps of providing a polynucleotide sequence comprising a polymorphism of the invention in a computer readable medium; and comparing the polymorphism containing polynucleotide sequence to at least one other polynucleotide or polypeptide sequence to identify identity (homology), i.e., screen for the presence of a polymorphism.

The invention also provides a kit comprising one or more of the herein-described polymorphic nucleic acids. The kit can include, e.g., a polynucleotide which includes one or more of the polymorphic sites described herein. The polynucleotide can be, e.g., a nucleotide sequence which includes one or more of the polymorphic sequences disclosed herein, or a fragment of the polymorphic sequence, as long as it includes the polymorphic site. The polynucleotide may alternatively contain a nucleotide sequence which includes a sequence complementary to one or more of the above noted polymorphic sequences, or a fragment of the complementary nucleotide sequence, provided that the fragment includes a polymorphic site in the polymorphic sequence.

Alternatively, or in addition, the kit can include an allele-specific oligonucleotide that hybridizes to a target nucleic acid containing a polymorphic site or a fragment thereof.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the SNPs of the invention may be used as genetic markers in linkage studies. This particularly applies to the polymorphisms at 1,731,127; 1,732,472; 1,865,017 and/or 2,195,995 because of their relatively high frequency.

Further scope of the applicability of the present invention will become apparent from the detailed description provided below.

It should be understood, however, that the following detailed description and examples, while indicating preferred embodiments of the invention, are given by way of illustration only, since various changes and modifications within the spirit and scope of the invention will become apparent to those skilled in the art from the following detailed description.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

### BRIEF DESCRIPTION OF THE DRAWINGS

FIG. 1 is the schematic depiction of the chromosomal structure of the human mGluR8 gene indicating the introns (1-8) and exons (1-10). Black or dark boxes represent coding exons (1-10) and the light boxes represent the non-coding exon (exon 10) and the nucleotides in the newly identified alleles are indicated.

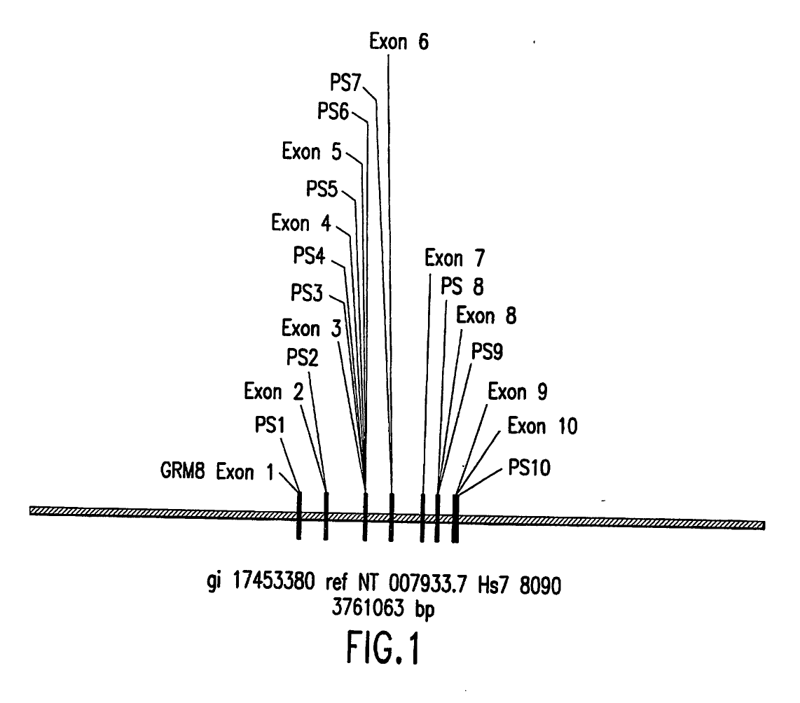

FIG. 2 depicts the RFLP analysis of PS6.

SEQ ID NO:1 is the reference (genomic) nucleotide sequence derived from/corresponds NT-007933.7 updated Dec. 10, 2001.

SEQ ID NO:2 is the nucleotide sequence corresponding to the published cDNA/mRNA sequence corresponding to XM045464 except that has an additional 52 bases at the 3′ end, which includes PS10.

SEQ. ID NO:3 is the reference (published) amino acid sequence of human mGluR8 receptor protein corresponding to XM045464.

SEQ ID NO:4 represents the nucleotide sequence of a single nucleotide polymorphism (PS3) in the mGluR8 gene that results in an amino acid change Ile 265 Thr.

SEQ ID NO:5 is the amino acid sequence of a variant mGluR8 receptor protein encoded by the SNP designated PS3.

SEQ ID NO:6 represents the nucleotide sequence of a single nucleotide polymorphism (PS6) in the mGluR8 gene that results in an amino acid change Phe 362 Tyr (F265Y).

SEQ ID NO:7 is the amino acid sequence of a variant mGluR8 receptor protein encoded by the SNP designated PS6.

SEQ ID NO:8 represents the nucleotide sequence of a single nucleotide polymorphism (PS9) in the mGluR8 gene that results in an amino acid change Pro 512 ala (P512A).

SEQ ID NO:9 is the amino acid sequence of a variant mGluR8 receptor protein encoded by the SNP designated PS9.

SEQ ID NO: 10 represents a single nucleotide polymorphism within a consensus sequence for the splice junction at the 5′ end of exon 6. Consequently, the presence of this SNP results in the translation of a shorter mGluR8 receptor polypeptide relative to normal/wild type.

SEQ ID NO:11 is the deduced amino acid sequence of SEQ ID NO:10.

### DETAILED DESCRIPTION OF THE INVENTION

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the methodologies, vectors etc which are reported in the publications that might be used in connection with the invention. Nothing herein is to be construed as an admission that the invention is not entitled to antedate such disclosure by virtue of prior invention.

In the description that follows, a number of terms used in the field of recombinant DNA technology are extensively utilized. In order to provide a clearer and consistent understanding of the specification and claims, including the scope to be given such terms, the following definitions are provided.

Definitions

The terms and abbreviations used in this document have their normal meanings unless otherwise designated. For example “_C” refers to degrees Celsius; “N” refers to normal or normality; “rnM” refers to millimole or millimoles; “g” refers to gram or grams; “ml” means milliliter or milliliters; “M” refers to molar or molarity; “.mu.g” refers to microgram or micrograms; and “μl” refers to microliter or microliters.

The term “gene” as used herein is intended to refer to a nucleic acid sequence which encodes a polypeptide. This definition includes various sequence polymorphisms, mutations, and/or sequence variants wherein such alterations do not affect the function of the gene product. The term “gene” is intended to include not only coding sequences but also regulatory regions such as promoters, enhancers, termination regions and similar untranslated nucleotide sequences. The term further includes all introns and other DNA sequences spliced from the mRNA transcript, along with variants resulting from alternative splice sites. A gene can be either RNA or DNA.

As used herein, the terms “DNA” and “DNAs” are defined as molecules comprising deoxyribonucleotides linked in standard 5′ to 3′ phosphodiester linkage, including both smaller oligodeoxyribonucleotides and larger deoxyribonucleic acids.

“Base Pair” refers to a partnership of adenine (A) with thymine (T), or of cytosine (C) with guanine (G) in a double-stranded DNA molecule. In RNA, uracil (U) is substituted for thymine. Base pairs are said to be “complementary” when their component bases pair up normally when a DNA or RNA molecule adopts a double-stranded configuration.

The term “Isoform” is intended to mean a particular form of a gene, mRNA, cDNA or the protein encoded thereby, distinguished from other forms by its particular sequence and/or structure.

Likewise, “Isogene” refers to one of the isoforms of a gene found in a population. An isogene contains all of the polymorphisms present in the particular isoform of the gene.

As used herein “nucleic acid” “nucleic acid molecule” “nucleic acid molecule” and “oligonucleotide” are used interchangeably and refer to a polymeric (2 or more monomers) form of nucleotides of any length, either ribonucleotides or deoxyribonucleotides. Oligonucleotides can be naturally occurring or synthetic. This term refers only to the primary structure of the molecule. Thus, this term includes double- and single stranded DNA and RNA. Nucleic acid molecules include both sense and antisense strands.

Nucleic acid molecule(s) generally refers to any polyribonucleotide or polydeoxyribonucleotide, which may be unmodified RNA or DNA or modified RNA or DNA. Thus, for instance, nucleic acid molecules as used herein refers to, among others, single- and double-stranded DNA, DNA that is a mixture of single- and double-stranded regions or single-, double- and triple-stranded regions, single- and double-stranded RNA, and RNA that is mixture of single- and double-stranded regions.

Preferred nucleic acid molecules of the invention include segments of DNA, or their complements including any one of the polymorphic sites shown in SEQ ID NOs:1 or 2. The segments are usually between 5 and 100 contiguous bases, and often range from 5, 10, 12, 15, 20, or 25 nucleotides to 10, 15, 30, 25, 20, 50 or 100 nucleotides. Nucleic acids between 5-10, 5-20, 10-20, 12-30, 15-30, 10-50, 20-50 or 20-100 bases are common. The polymorphic site can occur within any position of the segment.

“Nucleotide” refers to a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose, it is referred to as a nucleotide.

“Nucleic acid sequence” and its grammatical equivalents as used herein refers to an oligonucleotide, nucleotide, or nucleic acid molecule, and fragments or portions thereof, and to DNA or RNA of genomic or synthetic origin which may be single- or double-stranded, and represent the sense or antisense strand.

For brevity in this application, the symbol T is used to represent both thymidine in DNA and uracil in RNA. Thus, in RNA oligonucleotides, the symbol T should be construed to indicate a uracil residue.

All nucleic acid sequences, unless otherwise designated, are written in the direction from the 5′ end to the 3′ end, frequently referred to as “5′ to 3′”.

An “isolated nucleic acid” means an object species invention that is the predominant species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

“Intron(s)” and “Exon(s)”—Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease.

“Sequence” means the linear order in which monomers occur in a polymer, for example, the order of amino acids in a polypeptide or the order of nucleotides in a nucleic acid molecule.

“Amino acid sequence” as used herein refers to an oligopeptide, peptide, polypeptide, or protein sequence, and fragments or portions thereof, and to naturally occurring or synthetic molecules.

All amino acid or protein sequences, unless otherwise designated, are written commencing with the amino terminus (“N-terminus”) and concluding with the carboxy terminus (“C-terminus”).

Variations in polypeptide sequence will be referred to as follows: original amino acid (using 1 or 3 letter nomenclature), position, new amino acid. For (a hypothetical) example “R343Q” or “Arg343Gln” means that at position 343 an arginine (R) has been changed to glutamine (Q). Multiple mutations in one polypeptide will be shown between square brackets with individual mutations separated by commas.

“Isolated amino acid sequence” refers to any amino acid sequence, however constructed or synthesized, which is locationally distinct from the naturally occurring sequence.

“Isolated DNA sequence” “isolated nucleic acid sequence” refers to any DNA sequence, however constructed or synthesized, which is locationally distinct from its natural location in genomic DNA.

The term “reading frame” means the nucleotide sequence from which translation occurs “read” in triplets by the translational apparatus of transfer RNA (tRNA) and ribosomes and associated factors, each triplet corresponding to a particular amino acid. A frameshift mutation occurs when a base pair is inserted or deleted from a DNA segment. When this occurs, the result is a different protein from that coded for by the DNA segment prior to the frameshift mutation. To insure against this, the triplet codons corresponding to the desired polypeptide must be aligned in multiples of three from the initiation codon, i.e., the correct “reading frame” being maintained.

“Gene therapy” means the introduction of a functional gene or genes from some source by any suitable method into a living cell to correct for a genetic defect.

“Reference sequence” means SEQ ID NO: 1 (published genomic sequence of human mGluR8 gene NT 007933 published Feb. 9, 2001, updated Dec. 10, 2001 as NT 007933.7); SEQ ID NO:2 (published cDNA/mRNA sequence encoding human mGluR8 receptor protein) and/or SEQ ID NO:3 (published amino acid sequence of a mature mGluR8 receptor protein corresponding to the cDNA sequence of SEQ ID NO:2). The position of the polymorphisms relative to the reference mRNA sequence corresponding to SEQ ID NO:2 are detailed in Table 3. Table 2 lists the positions of the exons relative to the updated reference sequence (NT 007933.7).

The terms “complementary” or “complementarity”, as used herein, refer to the natural binding of nucleic acid molecules under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA molecules. Thus, a complementary nucleotide sequence refers to a sequence of nucleotides in a single-stranded molecule of DNA or RNA that is sufficiently complementary to another single strand to specifically (non-randomly) hybridize to it with consequent hydrogen bonding.

The term “hybridization” as used herein refers to a process in which a strand of nucleic acid joins with a complementary strand through base pairing. The conditions employed in the hybridization of two non-identical, but very similar, complementary nucleic acids varies with the degree of complementarity of the two strands and the length of the strands. Such techniques and conditions are well known to practitioners in this field.

Hybridization probes are capable of binding in a base-specific manner to a complementary strand of nucleic acid. Such probes include nucleic acids, peptide nucleic acids, as described in Nielsen et al., Science 254, 1497-1500 (1991). Hybridizations are usually performed under stringent conditions, for example, at a salt concentration of no more than 1M and a temperature of at least 25° C. For example, conditions of 5×SSPE (750 mM NaCl, 50 mM NaPhosphate, 5 mM EDTA, pH 7.4) and a temperature of 25°-30° C. are suitable for allele-specific probe hybridizations.

The term “stringency” refers to a set of hybridization conditions which may be varied in order to vary the degree of nucleic acid hybridization with another nucleic acid. (See the definition of “hybridization”, supra.)

Stringency of hybridization is used herein to refer to conditions under which polynucleic acid hybrids are stable. As known to those of skill in the art, the stability of hybrids is reflected in the melting temperature (Tₘ) of the hybrids. Tₘ can be approximated by the formula:
81.5° C.−16.6(log₁₀[Na⁺])+0.41(% G+C)−600/I,
where l is the length of the hybrids in nucleotides.

Tₘ decreases approximately 1-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions. As used herein:
(1) HIGH STRINGENCY conditions, with respect to fragment hybridization, refer to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhardt's solution, 5×SSPE, 0.2% SDS, 200 μg/ml denatured sonicated herring sperm DNA, at 42° C., followed by washing in 0.1×SSPE, and 0.1% SDS at 65° C.;
(2) MODERATE STRINGENCY conditions, with respect to fragment hybridization, refer to conditions equivalent to hybridization in 50% formamide, 5× Denhardt's solution, 5×SSPE, 0.2% SDS, 200 μg/ml denatured sonicated herring sperm DNA, at 42° C., followed by washing in 0.2×SSPE, 0.2% SDS, at 60° C.;
(3) LOW STRINGENCY conditions, with respect to fragment hybridization, refer to conditions equivalent to hybridization in 10% formamide, 5× Denhardt's solution, 6×SSPE, 0.2% SDS, 200 μg/ml denatured sonicated herring sperm DNA, followed by washing in 1×SSPE, 0.2% SDS, at 50° C.; and
(4) HIGH STRINGENCY conditions, with respect to oligonucleotide (i.e., synthetic DNA≦about 30 nucleotides in length) hybridization, refer to conditions equivalent to hybridization in 10% formamide, 5× Denhardt's solution, 6×SSPE, 0.2% SDS, 200 μg/ml denatured sonicated herring sperm DNA, at 42° C., followed by washing in 1×SSPE, and 0.2% SDS at 50° C.

It is understood that these conditions may be duplicated using a variety of buffers and temperatures and that they are not necessarily precise.

Denhardt's solution and SSPE (see, e.g., Sambrook et al. (1989) Molecular Cloning: A Laboratory Manual, 2nd Ed., Cold Spring Harbor Laboratory Press, Cold Spring Harbor, N.Y.) are well known to those of skill in the art as are other suitable hybridization buffers. For example, SSPE is pH 7.4 phosphate-buffered 0.18M NaCl. SSPE can be prepared, for example, as a 20× stock solution by dissolving 175.3 g of NaCl, 27.6 g of NaH₂PO₄ and 7.4 g EDTA in 800 ml of water, adjusting the pH to 7.4, and then adding water to 1 liter. Denhardt's solution (see, Denhardt (1966) Biochem. Biohphys. Res. Commun. 23:641) can be prepared, for example, as a 50× stock solution by mixing 5 g Ficoll (Type 400, Pharmacia LKB Biotechnology, INC., Piscataway N.J.), 5 g of polyvinylpyrrolidone, 5 g bovine serum albumin (Fraction V; Sigma, St. Louis Mo.) water to 500 ml and filtering to remove particulate matter.

The term “PCR” as used herein refers to the widely-known polymerase chain reaction employing a thermally-stable polymerase.

A “primer” is a nucleic acid fragment which functions as an initiating substrate for enzymatic or synthetic elongation. Thus, the term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified. The primers used for PCR of the exons are listed in Table 4.

A “probe” as used herein is a nucleic acid compound or a fragment thereof which hybridizes with any one of the herein disclosed nucleotide sequences.

“Polymorphism” refers to a variation in nucleotide sequence (and encoded polypeptide sequence, if relevant) at a given position in the genome within a population. A polymorphism is thus said to be “allelic,” in that, due to the existence of the polymorphism, some members of a species may have the unmutated sequence (i.e., the original “allele”) whereas other members may have a mutated sequence (i.e., the variant or mutant “allele”). For the purposes of this application, mutation as defined herein may represent a polymorphism.

“Polymorphic” refers to the condition in which two or more variants of a specific genomic sequence can be found in a population. A “polymorphic site” is the locus at which the variation occurs.

The term “allele” is used herein to refer to variants of a nucleotide sequence. A biallelic polymorphism has two forms. Typically the first identified allele is designated as the original allele whereas other alleles are designated as alternative alleles. Diploid organisms is homozygous or heterozygous for an allelic form.

As used herein, the term “SNP” or “SNP” includes all single base variants and also includes nucleotide insertions and deletions in addition to single nucleotide substitutions (e.g., A->G). Nucleotide substitutions are of two types. A transition is the replacement of one purine by another purine or one pyrimidine by another pyrimidine. A transversion is the replacement of a purine for a pyrimidine or vice versa.). A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998).

Typically, between different genomes or between different individuals, the polymorphic site is occupied by two different nucleotides. SNPs occur at defined positions within genomes and can be used for gene mapping, defining population structure, and performing functional studies. SNPs are useful as markers because many known genetic diseases are caused by point mutations and insertions/deletions. The conformation of the nucleic acid molecule is generally detectable, identifiable and/or distinguishable using methods known in the art, such as electrophoretic mobility as measured by gel electrophoresis, capillary electrophoresis, and/or susceptibility to endonuclease digestion etc.

The SNP (SNP) at nucleotide residue 1,732,472 is in a very important region of the mGluR8 gene. The SNP is within a consensus sequence for the splice junction at the 5′ end of exon 6. The presence of the SNP at this position implies that exon 6 may be skipped thereby resulting in the translation of a much shorter polypeptide relative to normal. Refer to SEQ ID NOs 10 and 11, representing the nucleotide and deduced amino acid sequence respectively.

A disease-related gene is any gene that, in one or more variant is associated with, or causative of, disease.

The term “genotype” as used herein refers the identity of the alleles present in an individual or a sample. The term “genotyping” a sample or an individual for an allelic marker consists of determining the specific allele or the specific nucleotide carried by an individual at an allelic marker.

The term “haplotype” refers to a combination of alleles present in an individual or a sample.

“Linkage” describes the tendency of genes, alleles, loci or genetic markers to be inherited together as a result of their location on the same chromosome, and can be measured by percent recombination between the two genes, alleles, loci or genetic markers. Loci occurring within 50 centimorgan of each other are linked. Some linked markers occur within the same gene or gene cluster.

“Linkage disequilibrium” or “allelic association” means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the haplotype ac to occur with a frequency of 0.25 in a population of individuals. If ac occurs more frequently, then alleles a and c are in linkage disequilibrium. Linkage disequilibrium may result from natural selection of certain combination of alleles or because an allele has been introduced into a population too recently/to have reached equilibrium with linked alleles.

A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected to indicate susceptibility to the disease in circumstances in which the gene Y may not have been identified or may not be readily detectable. Younger alleles (i.e., those arising from mutation relatively late in evolution) are expected to have a larger genomic sequencement in linkage disequilibrium. The age of an allele can be determined from whether the allele is shared between ethnic human groups and/or between humans and related species.

“Genetic variant” or “variant” means a specific genetic variant which is present at a particular genetic locus in at least one individual in a population and that differs from a reference sequence.

As used herein, the terms “genetic predisposition”, genetic susceptibility” and “susceptibility” all refer to the likelihood that an individual subject will develop a particular disease, condition or disorder. For example, a subject with an increased susceptibility or predisposition will be more likely that average to develop a disease, while a subject with a decreased predisposition will be less likely than average to develop the disease. Alternatively, a genetic variant is associated with an altered susceptibility or predisposition if the allele frequency of the genetic variant in a population or subpopulation with a disease, condition or disorder varies from its allele frequency in the population without the disease, condition or disorder (control population) or a reference sequence (wild type) by at least 1%, preferably by at least 2%, more preferably by at least 4% and more preferably still by at least 8%.

The term human includes both a human having or suspected of having a mGluR8-mediated disease and an asymptomatic human who may be tested for predisposition or susceptibility to such disease. At each position the human may be homozygous for an allele or the human may be a heterozygote.

A “patient” refers to a mammal in which modulation of an metabotropic glutamate receptor will have a beneficial effect. Patients in need of treatment involving modulation of metabotropic glutamate receptors can be identified using standard techniques known to those in the medical profession. Preferably, a patient is a human having a disease or disorder characterized by one or more of the following: (1) abnormal metabotropic glutamate receptor activity; (2) an abnormal level of a messenger whose production or secretion is affected by metabotropic glutamate receptor activity; and (3) an abnormal level or activity of a messenger whose function is affected by metabotropic glutamate receptor activity.

By “therapeutically effective amount” is meant an amount of an agent which relieves to some extent one or more symptoms of the disease or disorder in the patient; or returns to normal either partially or completely one or more physiological or biochemical parameters associated with or causative of the disease.

By “comprising” it is meant including, but not limited to, whatever follows the word “comprising”. Thus use of the term indicates that the listed elements are required, but that other elements are optional and may or may not be present. By “consisting essentially of” is meant that the listed elements are required, but that other elements are optional and may or may not be present depending upon whether or not they affect the activity or action of the listed elements.

The capacity to diagnose disease is of central concern to human, animal and plant genetic studies, and particularly to inherited disease diagnostics. Genetic disease diagnosis typically is pursued by analyzing variations in DNA sequences that distinguish genomic DNA among members of a population.

I. Novel Polymorphisms of the Invention

The present application provides 10 polymorphisms, specifically single nucleotide polymorphic sites in the mGluR8 gene. The polymorphic sites (sequences) identified by the inventors are referred to as PS1-10, which essentially represent novel allelic variants of the mGluR8 gene. Refer to Table 5 for each respective SNP, e.g., list of PS1-10.

Briefly, PS1 refers to a thymine (T) at position 1,392,239; cytosine (C) at position 1,528,555 (PS2); cytosine (C) at position 1,730,468 (PS3); guanine (G) at position 1,730,897 (PS4); guanine (G) at position 1,731,127 (PS5); adenine (A) at position 1,732,472 (PS6); adenine (A) at position 1,865,017(PS7); cytosine (C) at position 2,101,189(PS8); guanine (G) at position 2,101,237(PS9); cytosine (C) at position 2,195,995(PS10) with reference to SEQ ID NO:1.

II. Analysis of Polymorphisms

A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

Many of the methods described below require amplification of DNA from target samples. This can be accomplished by e.g., PCR. See generally PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

B. Detection of Polymorphisms in Target DNA

Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR.

Another type of analysis is sometimes referred to as de novo characterization. This analysis compares target sequences in different individuals to identify points of variation, i.e., polymorphic sites. By analyzing a groups of individuals representing the greatest ethnic diversity among humans and greatest breed and species variety in plants and animals, patterns characteristic of the most common alleles/haplotypes of the locus can be identified, and the frequencies of such populations in the population determined. Additional allelic frequencies can be determined for subpopulations characterized by criteria such as geography, race, or gender. Yet another type of analysis proposes determining which form(s) of a characterized polymorphism are present in individuals under test. Several of the methods known in the art for detecting specific single nucleotide polymorphisms are summarized below. The method of the present invention is understood to include all available methods.

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals.

Consequently, an embodiment of the invention proposes the design of appropriate probes that hybridize to a specific gene of the mGluR8 gene. The genomic DNA sequences for human mGluR8 has been published and corresponds to SEQ ID NO: 1. Alternatively, these probes may incorporate other regions of the relevant genomic locus, including intergenic sequences.

Importantly, the polymorphic nucleotide sequences of the invention, i.e., PS1-PS10 may be used for their ability to selectively form duplex molecules with complementary stretches of human chromosome 7q31.3-q32.1 or cDNAs from that region or to provide primers for amplification of DNA or cDNA from this region. The design of additional oligonucleotides for use in the amplification and detection of mGluR8 polymorphic alleles by the method of the invention is facilitated by the availability of both updated sequence information from human chromosome 7q31.3-q32.1.—which contains the human GluR locus, and updated human polymorphism information available for this locus. For example, the genomic DNA sequence for the mGluR8 receptor is shown in SEQ ID NO: 1. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher.

The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are also envisioned for certain embodiments. Lengths of oligonucleotides of at least about 18 to 20 nucleotides are well accepted by those of skill in the art as sufficient to allow sufficiently specific hybridization so as to be useful as a molecular probe. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50° C. to about 70° C. Such selective conditions may tolerate little, if any, mismatch between the probe and the template or target strand.

Thus, oligonucleotides which are complementary to and hybridizable with any portion of the novel polymorphic sequences (SNPs) disclosed herein also are contemplated for therapeutic use. See U.S. Pat. No. 5,639,595, wherein methods of identifying oligonucleotide sequences that display in vivo activity are thoroughly described.

Indeed, an alternative embodiment of the invention provides a method for determining if a sequence polymorphism is the present in a subject, such as a human. The method includes providing a nucleic acid from the subject and contacting the nucleic acid with an oligonucleotide that hybridizes to any one or more of the polymorphic sequences selected from the group consisting of PS1-PS10. Hybridization between the nucleic acid and the oligonucleotide is then determined. Hybridization of the oligonucleotide to the nucleic acid sequence indicates the presence of the polymorphism in said subject.

2. Nucleic Acid Arrays

The polymorphisms can also be identified by hybridization to nucleic acid arrays, some example of which are described by WO 95/11995 (incorporated by reference in its entirety for all purposes). See also WO 92/10588 to Fodor et al., which discloses a process for sequencing, fingerprinting, and mapping nucleic acids by hybridization to an array of oligonucleotides. Detection involves positional localization of the region where hybridization has taken place. See also U.S. Pat. Nos. 5,324,633 and 5,424,186 to Fodor et al., U.S. Pat. Nos. 5,143,854 and 5,405,783 to Pirrung et al., WO 90/15070 to Pirrung et al., Pease et al., “Light-generated Oligonucleotide Arrays for Rapid DNA Sequence Analysis”, Proc. Natl. Acad. Sci. USA 91: 5022-26 (1994), Beattie et al., “Advances in—Genosensor Research,” Clin. Chem. 41(5): 700-09 (1995), and Landegren et al., “Reading Bits of Genetic Information: Methods for Single-Nucleotide Polymorphism Analysis,” Genome Research, 8:769-776 (1998) all of which are suitable for the present invention.

3. Allele-Specific Primers

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer. See, e.g., WO 93/22456.

4. Direct-Sequencing

Any of a variety of sequencing reactions known in the art can be used to directly sequence the allele. Exemplary sequencing reactions include those based on techniques developed by Maxim and Gilbert ((1977) Proc. Natl. Acad. Sci. USA 74:560) or Sanger (Sanger et al (1977) Proc. Nat. Acad. Sci. USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

6. Single-Strand Conformation Polymorphism Analysis

In other embodiments, alterations in electrophoretic mobility will be used to identify a mGluR8 polymorphism. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control mGluR8 locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

7. Another method proposes the use of a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

8. In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an mGluR8 locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

9. Alternatively, a solution-based method can be used for determining the identity of the nucleotide of a polymorphic site. Cohen, D. et al. (French Patent 2,650,840; PCT Appln. No. WO91/02087). Herein, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

Several primer-guided nucleotide incorporation procedures for assaying polymorphic sites in DNA have also been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

10. For mutations that produce premature termination of protein translation, the protein truncation test (PTT) offers an efficient diagnostic approach (Roest, et. al., (1993) Hum. Mol Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g., blood, obtained by known techniques (e.g. venipuncture) or saliva. Alternatively, nucleic acid tests can be performed on dry samples (e.g. hair or skin). When using RNA or protein, the cells or tissues that may be utilized must express a mGluR8 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

11. In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand.

12. Examples of other techniques for detecting alleles include, but are not limited to, selective oligonucleotide hybridization, selective amplification, or selective primer extension. An exemplary embodiment proposes preparing oligonucleotide primers in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

III. Methods of Use

After determining polymorphic form(s) present in an individual at one or more polymorphic sites, this information can be used in a number of methods.

1. Correlation of Polymorphisms with Phenotypic Traits

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components. Phenotypic traits include symptoms of, or susceptibility to mGluR8 mediated diseases of which a component is or may be genetic, such as diseases of the nervous system exemplified by schizophrenia, and other mGluR8-mediated diseases such as Parkinson's disease, Alzheimer's disease, Huntington's disease, stroke, anxiety, cognitive dysfunction, attention deficit hyperactivity disorder, autism, pain and inflammation.

Correlation is performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods such as a kappa.-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient.

2. Genetic Mapping of Phenotypic Traits

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992) (each of which is incorporated by reference in its entirety for all purposes).

Linkage analysis, is based upon establishing a correlation between the transmission of genetic markers and that of a specific trait throughout generations within a family. In this approach, all members of a series of affected families are genotyped with a few hundred markers. By comparing genotypes in all family members, one can attribute sets of alleles to parental haploid genomes (haplotyping or phase determination). The origin of recombined fragments is then determined in the offspring of all families. Those that co-segregate with the trait are tracked. After pooling data from all families, statistical methods are used to determine the likelihood that the marker and the trait are segregating independently in all families. As a result of the statistical analysis, one or several regions are selected as candidates, based on their high probability to carry a trait causing allele. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction .theta., versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (.theta.), ranging from .theta=0.0 (coincident loci) to .theta.=0.50 (unlinked). Thus, the likelihood at a given value of theta. is: probability of data if loci linked at theta. to probability of data if loci unlinked. The computed likelihood are usually expressed as the log.sub.10 of this ratio (i.e., a LOD score). Thus, a LOD score of 3 indicates 1000:1 odds against an apparent observed linkage being a coincidence. The use of logarithms allows data collected from different families to be combined by simple addition. Computer programs are available for the calculation of LOD scores for differing values of .theta. (e.g., LIPED, MLINK (Lathrop, Proc. Nat. Acad. Sci. (USA) 81, 3443-3446 (1984)). For any particular LOD score, a recombination fraction may be determined from mathematical tables. See Smith et al., Mathematical tables for research workers in human genetics (Churchill, London, 1961); Smith, Ann. Hum. Genet. 32, 127-150 (1968). The value of theta. at which the LOD score is the highest is considered to be the best estimate of the recombination fraction.

Positive LOD score values suggest that the two loci are linked, whereas negative values suggest that linkage is less likely (at that value of .theta.) than the possibility that the two loci are unlinked. By convention, a combined LOD score of +3 or greater (equivalent to greater than 1000:1 odds in favor of linkage) is considered definitive evidence that two loci are linked. Similarly, by convention, a negative LOD score of −2 or less is taken as definitive evidence against linkage of the two loci being compared. Negative linkage data are useful in excluding a chromosome or a segment thereof from consideration. The search focuses on the remaining non-excluded chromosomal locations.

In addition one of skill in the art can readily identify other alleles (including polymorphisms and mutations) that are in linkage disequilibrium with an allele associated with a disease or disorder. For example, a nucleic acid sample from a first group of subjects without a particular disorder can be collected, as well as DNA from a second group of subjects with the disorder. The nucleic acid sample can then be compared to identify those alleles that are over-represented in the second group as compared with the first group, wherein such alleles are presumably associated with a disorder, which is caused or contributed to by inappropriate mGluR8 regulation.

The organization of single nucleotide variations (polymorphisms) in the primary sequence of a gene into one of the limited number of combinations that exist as units of inheritance is termed a haplotype. Each haplotype therefore contains significantly more information than individual unorganized polymorphisms. Haplotypes provide an accurate measurement of the genomic variation in the two chromosomes of an individual. It is well-established that many diseases are associated with specific variations in gene sequences. However while there are examples in which individual polymorphisms act as genetic markers for a particular phenotype, in other cases an individual polymorphism may be found in a variety of genomic backgrounds and therefore shows no definitive coupling between the polymorphism and the causative site for the phenotype (Clark A G et al. 1998 Am J Hum Genet 63:595-612; Ulbrecht M et al. 2000 Am Jrespir Crit Care Med 161: 469-74). In addition, the marker may be predictive in some populations, but not in other populations (Clark A G et al. 1998 supra). In these instances, a haplotype will provide a superior genetic marker for the phenotype (Clark A G et al. 1998 supra; Ulbrecht M et al. 2000, supra; Ruano G & Stephens J C Gen EngNews 19 (21), December 1999).

Analysis of the association between each observed haplotype and a particular phenotype permits ranking of each haplotype by its statistical power of prediction for the phenotype. Haplotypes found to be strongly associated with the phenotype can then have that positive association confirmed by alternative methods to minimize false positives. For a gene suspected to be associated with a particular phenotype, if no observed haplotypes for that gene show association with the phenotype of interest, then it may be inferred that variation in the gene has little, if any, involvement with that phenotype guano & Stephens 1999, supra). Thus, information on the observed haplotypes and their frequency of occurrence in various population groups will be useful in a variety of research and clinical applications.

IV. Modified Polypeptides and Gene Sequences

The invention further provides variant forms of nucleic acids and corresponding proteins. The nucleic acids described herein are designated PS1-PS10. Corresponding variant proteins encoded by each are also included.

Consequently, in one embodiment, the invention provides an isolated polynucleotide comprising a polymorphic variant of the mGluR8 gene or a fragment of the gene which contains at least one of the novel polymorphic sites described herein. The nucleotide sequence of a variant mGluR8 gene is identical to the reference genomic sequence for those portions of the gene examined, as described in the Examples below, except that it comprises a different nucleotide at one or more of the novel polymorphic sites PS1-PS10. Similarly, the nucleotide sequence of a variant fragment of the mGluR8 gene is identical to the corresponding portion of the reference sequence except for having a different nucleotide at one or more of the novel polymorphic sites described herein. Thus, the invention specifically does not include polynucleotides comprising a nucleotide sequence identical to the reference sequence (or other reported mGluR8 sequences) or to portions of the reference sequence (or other reported mGluR8 sequences), except for genotyping oligonucleotides as described elsewhere in the application.

The location of a polymorphism in a variant gene or fragment is identified by aligning its sequence against SEQ ID NO:1 when considering a variant polypeptide encoded by any one of the polymorphic sequences disclosed herein. The polymorphism is selected from the group consisting of thymine at PS1, cytosine at PS2, cytosine at PS3, guanine at PS4, guanine at PS5, adenine at PS6, adenine at PS7, cytosine at PS8, guanine at PS9, and cytosine at PS10.

Polymorphic variants of the invention may be prepared by isolating a clone containing the mGluR8 gene from a human genomic library. The clone may be sequenced to determine the identity of the nucleotides at the polymorphic sites described herein. Any particular variant claimed herein could be prepared from this clone by performing in vitro mutagenesis using procedures well-known in the art. mGluR8 isogenes may be isolated using any method that allows separation of the two “copies” of the mGluR8 gene present in an individual, which, as readily understood by the skilled artisan, may be the same allele or different alleles. Separation methods include targeted in vivo cloning (TIVC) in yeast as described in WO 98/01573, U.S. Pat. No. 5,866,404, and U.S. Pat. No. 5,972,614. Another method, which is described in U.S. Pat. No. 5,972,614, uses an allele specific oligonucleotide in combination with primer extension and exonuclease degradation to generate hemizygous DNA targets. Yet other methods are single molecule dilution (SMD) as described in Ruano et al., Proc. Natl. Acad. Sci. 87:6296-6300, 1990; and allele specific PCR (Ruano et al., 17 Nucleic Acids. Res. 8392, 1989; Ruano et al., 19 Nucleic Acids Res. 6877-6882, 1991; Michalatos-Beloin et al., 24 Nucleic Acids Res. 4841-4843, 1996).

The invention also provides mGluR8 genome anthologies, which are collections of mGluR8 isogenes found in a given population. The population may be any group of at least two individuals, including but not limited to a reference population, a population group, a family population, a clinical population, and a same sex population.

An mGluR8 genome anthology may comprise individual mGluR8 isogenes stored in separate containers such as microtest tubes, separate wells of a microtitre plate and the like. Alternatively, two or more groups of the mGluR8 isogenes in the anthology may be stored in separate containers. Individual isogenes or groups of isogenes in a genome anthology may be stored in any convenient and stable form, including but not limited to in buffered solutions, as DNA precipitates, freeze-dried preparations and the like.

An isolated polynucleotide containing a polymorphic variant nucleotide sequence of the invention may be operably linked to one or more expression regulatory elements in a recombinant expression vector capable of being propagated and expressing the encoded mGluR8 protein in a prokaryotic or a eukaryotic host cell. Examples of expression regulatory elements which may be used include, but are not limited to, the lac system, operator and promoter regions of phage lambda, yeast promoters, and promoters derived from vaccinia virus, adenovirus, retroviruses, or SV40.

Other regulatory elements include, but are not limited to, appropriate leader sequences, termination codons, polyadenylation signals, and other sequences required for the appropriate transcription and subsequent translation of the nucleic acid sequence in a given host cell. Of course, the correct combinations of expression regulatory elements will depend on the host system used.

In addition, it is understood that the expression vector contains any additional elements necessary for its transfer to and subsequent replication in the host cell. Examples of such elements include, but are not limited to, origins of replication and selectable markers. Such expression vectors are commercially available or are readily constructed using methods known to those in the art (e.g., F. Ausubel et al., 1987, in “Current Protocols in Molecular Biology”, John Wiley and Sons, New York, N.Y.).

Host cells which may be used to express the variant mGluR8 sequences of the invention include, but are not limited to, eukaryotic and mammalian cells, such as animal, plant, insect and yeast cells, and prokaryotic cells, such as E. coli, or algal cells as known in the art. The recombinant expression vector may be introduced into the host cell using any method known to those in the art including, but not limited to, microinjection, electroporation, particle bombardment, transduction, and transfection using DEAE dextran, lipofection, or calcium phosphate (see e.g., Sambrook et al. (1989) in “Molecular Cloning. A Laboratory Manual”, Cold Spring Harbor Press, Plainview, N.Y.).

In a preferred aspect, eukaryotic expression vectors that function in eukaryotic cells, and preferably mammalian cells, are used. Non-limiting examples of such vectors include vaccinia virus vectors, adenovirus vectors, herpes virus vectors, and baculovirus transfer vectors. Preferred eukaryotic cell lines include COS cells, CHO cells, HeLa cells, NIH/3T3 cells, and embryonic stem cells (Thomson, J. A. et al., 1998 Science 282:1145-1147). Particularly preferred host cells are mammalian cells.

As will be readily recognized by the skilled artisan, expression of polymorphic variants of the mGluR8 gene will produce mGluR8 mRNAs varying from each other at any polymorphic site retained in the spliced and processed mRNA molecules. These mRNAs can be used for the preparation of an mGluR8 cDNA comprising a nucleotide sequence which is a polymorphic variant of the mGluR8 reference coding sequence corresponding to SEQ ID NO:2.

Thus, the invention also provides mGluR8 mRNAs and corresponding cDNAs which comprise a nucleotide sequence that is substantially identical to SEQ ID NO:2, or its corresponding RNA sequence, except for having one or both polymorphisms selected from the group consisting of thymine at a position corresponding to nucleotide 357, cytosine at a position corresponding to nucleotide 693, cytosine at a position corresponding to nucleotide 794, adenine at a position corresponding to nucleotide 1095, and guanine at a position corresponding to nucleotide 1534.

Fragments of these variant mRNAs and cDNAs are included in the scope of the invention, provided they contain the novel polymorphisms described herein. The invention specifically excludes polynucleotides identical to previously identified and characterized mGluR8 cDNAs and fragments thereof.

Polynucleotides comprising a variant RNA or DNA sequence may be isolated from a biological sample using well-known molecular biological procedures or may be chemically synthesized. Genomic and cDNA fragments of the invention comprise at least one novel polymorphic site identified herein and have a length of at least 10 nucleotides and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

In describing the polymorphic sites identified herein, reference is made to the sense strand of the gene for convenience. However, as recognized by the skilled artisan, nucleic acid molecules containing the mGluR8 gene may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site.

Consequently, the invention also includes single-stranded polynucleotides which are complementary to the sense strand of the mGluR8 genomic variants described herein. Polynucleotides comprising a polymorphic gene variant or fragment may be useful for therapeutic purposes. For example, where a patient could benefit from expression, or increased expression, of a particular mGluR8 protein isoform, an expression vector encoding the isoform may be administered to the patient. The patient may be one who lacks the mGluR8 isogene encoding that isoform or may already have at least one copy of that isogene. In other situations, it may be desirable to decrease or block expression of a particular mGluR8 isogene. Expression of an mGluR8 isogene may be turned off by transforming a targeted organ, tissue or cell population with an expression vector that expresses high levels of untranslatable mRNA for the isogene.

Alternatively, oligonucleotides directed against the regulatory regions (e.g., promoter, introns, enhancers, 3′ untranslated region) of the isogene may block transcription. Oligonucleotides targeting the transcription initiation site, e.g., between positions −10 and +10 from the start site are preferred. Similarly, inhibition of transcription can be achieved using oligonucleotides that base-pair with region(s) of the isogene DNA to form triplex DNA (see e.g., Gee et al. in Huber, B. E. and B. I. Carr, Molecular and Immunologic Approaches, Futura Publishing Co., Mt. Kisco, N.Y., 1994).

# Antisense oligonucleotides may also be designed to block translation of mGluR8 mRNA transcribed from a particular isogene. It is also contemplated that ribozymes may be designed that can catalyze the specific cleavage of mGluR8 mRNA transcribed from a particular isogene. The oligonucleotides may be delivered to a target cell or tissue by expression from a vector introduced into the cell or tissue in vivo or ex vivo.

Alternatively, the oligonucleotides may be formulated as a pharmaceutical composition for administration to the patient. Oligoribonucleotides and/or oligodeoxynucleotides intended for use as antisense oligonucleotides may be modified to increase stability and half-life. Possible modifications include, but are not limited to phosphorothioate or 2′ O-methyl linkages, and the inclusion of nontraditional bases such as inosine and queosine, as well as acetyl-, methyl-, thio-, and similarly modified forms of adenine, cytosine, guanine; thymine, and uracil which are not as easily recognized by endogenous nucleases. The reader is directed to the following references on nucleic acid triplex formation and uses: Progress in developments of Triplex-Based strategies: Giovannangeli C; Helene C: Antisense and Nucleic Acid Drug Development/7/4 (413421)/1997; Recent developments in triple-helix regulation of gene expression: Neidle S: Anti-Cancer Drug Design/12/5 (433-442)/1997; Triplex DNA: Fundamentals, advances, and potential applications for gene therapy: Chan P P; Glazer P M: Journal of Molecular Medicine/75/4 (267-282)/1997; Oligonucleotide directed triple helix formation: Sun J-S; Garestier T; Helene C: Current Opinion in Structural Biology/6/3 (327-333)/1996; C Mayfield, M Squibb, D Miller (1994) Inhibition of nuclear protein binding to the human Ki-ras promoter by triplex-forming oligonucleotides Biochemistry 3 3,3 3 5 8-3 3 63; W M Olivas, L J Maher (1996) Binding of DNA oligonucleotides to sequences in the promoter of the human bc1-2 gene Nucleic Acids Research 24, 1758-1764; C Mayfield, S Ebinghaus, J Gees, D Jones, B Rodu, M Squibb, D Miller (1994) Triplex formation by the human HA-ras promoter inhibits Sp I binding and in vitro transcription J Biol Chem 269,18232-18238; and J E Gee, G R Revankar, T S Rao, M E Hogan (1995) Triplex formation at the rat neu gene utilizing imidazole and 2′-deoxy-6-thioguanosine base substitutions Biochemistry 34,2042-2048.

The invention also provides an isolated polypeptide comprising a polymorphic variant of the reference mGluR8 amino acid sequence shown in SEQ ID NO:3. The location of a variant amino acid in an mGluR8 polypeptide or fragment of the invention is identified by aligning its sequence against SEQ ID NO:3.

An mGluR8 protein variant of the invention comprises an amino acid sequence identical to SEQ ID NO:3 except for the amino acids described below. The invention specifically excludes amino acid sequences identical to those previously identified for mGluR8, including SEQ ID NO:3, and previously described fragments thereof. mGluR8 protein variants included within the invention comprise all amino acid sequences based on SEQ ID NO:3 and having threonine at a position corresponding to amino acid position 265, tyrosine at a position corresponding to amino acid position 362 and alanine at a position corresponding to amino acid position 512.

The invention also includes mGluR8 peptide variants, which are any fragments of an mGluR8 protein variant that contains at least one the aforementioned variant amino acids. An mGluR8 peptide variant is at least 6 amino acids in length and is preferably any number between 6 and 30 amino acids long, more preferably between 10 and 25, and most preferably between 15 and 20 amino acids long. Such mGluR8 peptide variants may be useful as antigens to generate antibodies specific for one of the above mGluR8 isoforms. In addition, the mGluR8 peptide variants may be useful in drug screening assays.

An mGluR8 variant protein or peptide of the invention may be prepared by chemical synthesis or by expressing one of the variant mGluR8 genomic and cDNA sequences as described above. Alternatively, the mGluR8 protein variant may be isolated from a biological sample of an individual having an mGluR8 isogene which encodes the variant protein. Where the sample contains two different mGluR8 isoforms (i.e., the individual has different mGluR8 isogenes), a particular mGluR8 isoform of the invention can be isolated by immunoaffinity chromatography using an antibody which specifically binds to that particular mGluR8 isoform but does not bind to the other mGluR8 isoform. The expressed or isolated mGluR8 protein may be detected by methods known in the art, including Coomassie blue staining, silver staining, and Western blot analysis using antibodies specific for the isoform of the mGluR8 protein as discussed further below.

mGluR8 variant proteins can be purified by standard protein purification procedures known in the art, including differential precipitation, molecular sieve chromatography, ion-exchange chromatography, isoelectric focusing, gel electrophoresis, affinity and immunoaffinity chromatography and the like. (Ausubel et. al., 1987, In Current Protocols in Molecular Biology John Wiley and Sons, New York, N.Y.). In the case of immunoaffinity chromatography, antibodies specific for a particular polymorphic variant may be used.

A polymorphic variant mGluR8 gene of the invention may also be fused in frame with a heterologous sequence to encode a chimeric mGluR8 protein. The non-mGluR8 portion of the chimeric protein may be recognized by a commercially available antibody. In addition, the chimeric protein may also be engineered to contain a cleavage site located between the mGluR8 and non-mGluR8 portions so that the mGluR8 protein may be cleaved and purified away from the non-mGluR8 portion.

An additional embodiment of the invention relates to using a novel mGluR8 protein isoform in any of a variety of drug screening assays. Such screening assays may be performed to identify agents that bind specifically to all known mGluR8 protein isoforms or to only a subset of one or more of these isoforms. The agents may be from chemical compound libraries, peptide libraries and the like. The mGluR8 protein or peptide variant may be free in solution or affixed to a solid support.

In one embodiment, high throughput screening of compounds for binding to an mGluR8 variant may be accomplished using the method described in PCT application WO84/03565, in which large numbers of test compounds are synthesized on a solid substrate, such as plastic pins or some other surface, contacted with the mGluR8 protein(s) of interest and then washed. Bound mGluR8 protein(s) are then detected using methods well-known in the art.

In another embodiment, a novel mGluR8 protein isoform may be used in assays to measure the binding affinities of one or more candidate drugs targeting the mGluR8 protein.

In another embodiment, the invention provides antibodies specific for and immunoreactive with one or more of the novel mGluR8 variant proteins described herein. The antibodies may be either monoclonal or polyclonal in origin. The mGluR8 protein or peptide variant used to generate the antibodies may be from natural or recombinant sources or produced by chemical synthesis using synthesis techniques known in the art. If the mGluR8 protein variant is of insufficient size to be antigenic, it may be conjugated, complexed, or otherwise covalently linked to a carrier molecule to enhance the antigenicity of the peptide. Examples of carrier molecules, include, but are not limited to, albumins 14 (e.g., human, bovine, fish, ovine), and keyhole limpet hemocyanin (Basic and Clinical Immunology, 1991, Eds. D. P. Stites, and A. I. Terr, Appleton and Lange, Norwalk Conn., San Mateo, Calif.).

The term “antibodies” is meant to include polyclonal antibodies, monoclonal antibodies, and the various types of antibody constructs such as for example F(ab)₂, Fab and single chain Fv. Antibodies are defined to be specifically binding if they bind at least one of the variant gene products of or synthetic products thereof. Affinity of binding can be determined using conventional techniques, for example those described by Scatchard et al., Ann. N.Y. Acad. Sci., 51:660 (1949).

Antibodies can be prepared using any suitable method. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. No. RE 32,011, U.S. Pat. Nos. 4,902,614, 4,543,439 and 4,411,993—Monoclonal Antibodies, Hybridomas: A New Dimension in Biological Analyses, Plenum Press, Kennett, McKearn, and Bechtol (eds.), (1980). The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 1:1-9 (1990) which is incorporated herein by reference.

Similarly, binding partners can be constructed using recombinant DNA techniques to incorporate the variable regions of a gene that encodes a specific binding antibody. Such a technique is described in Larrick et al., Biotechnology, 1: 394(1989). Once isolated and purified, the antibodies may be used to detect the presence of antigen in a sample using established assay protocols.

Consequently, an embodiment contemplates an antibody specific for an allelic variant of human mGluR8 receptor polypeptide, preferably one having threonine at an amino acid position corresponding to position 265, tyrosine at a position corresponding to amino acid position 362 and alanine at a position corresponding to amino acid position 512 in SEQ ID NO; 3 or a fragment(s) thereof comprising at least 10 amino acids provided that the fragment comprises the allelic variant at any one or more of the aforementioned positions.

The invention further provides a method of detecting the presence of a polypeptide having one or more amino acid residue polymorphisms in a subject. The method includes providing a protein sample from the subject and contacting the sample with the above-described antibody under conditions that allow for the formation of antibody-antigen complexes. The antibody-antigen complexes are then detected. The presence of the complexes indicates the presence of the variant polypeptide encoded by a variant polynucleotide substantially similar to any one polymorphic nucleotides sequences disclosed herein.

In one embodiment, an antibody specifically immunoreactive with one of the novel mGluR8 protein isoforms described herein is administered to an individual to neutralize activity of the mGluR8 isoform expressed by that individual.

The antibody may be formulated as a pharmaceutical composition which includes a pharmaceutically acceptable carrier. Antibodies specific for and immunoreactive with one of the novel mGluR8 protein isoform described herein may be used to immunoprecipitate the mGluR8 protein variant from solution as well as react with mGluR8 protein isoforms on Western or immunoblots of polyacrylamide gels on membrane supports or substrates.

In another preferred embodiment, the antibodies will detect mGluR8 protein isoforms in paraffin or frozen tissue sections, or in cells which have been fixed or unfixed and prepared on slides, coverslips, or the like, for use in immunocytochemical, immunohistochemical, and immunofluorescence techniques.

In another embodiment, an antibody specifically immunoreactive with one of the novel mGluR8 protein variants described herein is used in immunoassays to detect this variant in biological samples.

Effect(s) of the polymorphisms identified herein on expression of mGluR8 may be investigated by preparing recombinant cells and/or organisms, preferably recombinant animals, containing a polymorphic variant of the mGluR8 gene. As used herein, “expression” includes but is not limited to one or more of the following: transcription of the gene into precursor mRNA; splicing and other processing of the precursor mRNA to produce mature mRNA; mRNA stability; translation of the mature mRNA into mGluR8 protein (including codon usage and tRNA availability); and, glycosylation and/or other modifications of the translation product, if required for proper expression and function. To prepare a recombinant cell of the invention, the desired mGluR8 isogene may be introduced into the cell in a vector such that the isogene remains extrachromosomal. In such a situation, the gene will be expressed by the cell from the extrachromosomal location.

In a preferred embodiment, the mGluR8 isogene is introduced into a cell in such a way that it recombines with the endogenous mGluR8 gene present in the cell. Such recombination requires the occurrence of a double recombination event, thereby resulting in the desired mGluR8 gene polymorphism. Vectors for the introduction of genes both for recombination and for extrachromosomal maintenance are known in the art, and any suitable vector or vector construct may be used in the invention. Methods such as electroporation, particle bombardment, calcium phosphate co-precipitation and viral transduction for introducing DNA into cells are known in the art; therefore, the choice of method may lie with the competence and preference of the skilled practitioner. Examples of cells into which the mGluR8 isogene may be introduced include, but are not limited to, continuous culture cells, such as COS, NIH/3T3, and primary or culture cells of the relevant tissue type, i.e., they express the mGluR8 isogene. Such recombinant cells can be used to compare the biological activities of the different protein variants. Recombinant organisms, i.e., transgenic animals, expressing a variant mGluR8 gene are prepared using standard procedures known in the art.

Preferably, a construct comprising the variant gene is introduced into a nonhuman animal or an ancestor of the animal at an embryonic stage, i.e., the one cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells. Examples of animals into which the ILI3 16 isogenes may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W.H. Freeman and Company, New York, pages 254-272).

Transgenic animals stably expressing a human mGluR8 isogene and producing human mGluR8 protein can be used as biological models for studying diseases related to abnormal mGluR8 expression and/or activity, and for screening and assaying various candidate drugs, compounds, and treatment regimens to reduce the symptoms or effects of these diseases.

An additional embodiment of the invention relates to pharmaceutical compositions for treating disorders affected by expression or function of a novel mGluR8 isogene described herein.

The pharmaceutical composition may comprise any of the following active ingredients: a polynucleotide comprising one of these novel mGluR8 isogenes; an antisense oligonucleotide directed against one of the novel mGluR8 isogenes, a polynucleotide encoding such an antisense oligonucleotide, or another compound which inhibits expression of a novel mGluR8 isogene described herein.

Preferably, the composition contains the active ingredient in a therapeutically effective amount. By therapeutically effective amount is meant that one or more of the symptoms relating to disorders affected by expression or function of a novel mGluR8 isogene is reduced and/or eliminated. The composition also comprises a pharmaceutically acceptable carrier, examples of which include, but are not limited to, saline, buffered saline, dextrose, and water. Those skilled in the art may employ a formulation most suitable for the active ingredient, whether it is a polynucleotide, oligonucleotide, protein, peptide or small molecule antagonist.

The pharmaceutical composition may be administered alone or in combination with at least one other agent, such as a stabilizing compound. Administration of the pharmaceutical composition may be by any number of routes including, but not limited to oral, intravenous, intramuscular, intra-arterial, intramedullary, intrathecal, intraventricular, intradermal, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.).

For any composition, determination of the therapeutically effective dose of active ingredient and/or the appropriate route of administration is well within the capability of those skilled in the art. For example, the dose can be estimated initially either in cell culture assays or in animal models. The animal model may also be used to determine the appropriate concentration range and route of administration. Such information can then be used to determine useful doses and routes for administration in humans. The exact dosage will be determined by the practitioner, in light of factors relating to the patient requiring treatment, including but not limited to severity of the disease state, general health, age, weight and gender of the patient, diet, time and frequency of administration, other drugs being taken by the patient, and tolerance/response to the treatment. Information on the identity of genotypes and haplotypes for the mGluR8 gene of any particular individual as well as the frequency of such genotypes and haplotypes in any particular population of individuals is expected to be useful for a variety of basic research and clinical applications.

Thus, the invention also provides compositions and methods for detecting the novel mGluR8 polymorphisms identified herein. The compositions comprise at least one mGluR8 genotyping oligonucleotide. In one embodiment, an mGluR8 genotyping oligonucleotide is a probe or primer capable of hybridizing to a target region that is located close to, or that contains, one of the novel polymorphic sites described herein, supra.

V. Pharmacogenomics

Knowledge of the particular alleles associated with a susceptibility to developing a particular disease or condition, alone or in conjunction with information on other genetic defects contributing to the particular disease or condition allows a customization of the prevention or treatment in accordance with the individual's genetic profile, the goal of “pharmacogenomics”.

The invention further provides methods for assessing the Pharmacogenomic susceptibility of a subject harboring a single nucleotide polymorphism to a particular pharmaceutical compound, or to a class of such compounds. Genetic polymorphism in drug-metabolizing enzymes, drug transporters, receptors for pharmaceutical agents, and other drug targets have been correlated with individual differences based on distinction in the efficacy and toxicity of the pharmaceutical agent administered to a subject. Pharmacogenomic characterization of a subject's susceptibility to a drug enhances the ability to tailor a dosing regimen to the particular genetic constitution of the subject, thereby enhancing and optimizing the therapeutic effectiveness of the therapy. Thus, comparison of an individual's mGluR8 profile to the population profile for a vascular disorder, permits the selection or design of drugs or other therapeutic regimens that are expected to be safe and efficacious for a particular patient or patient population (i.e., a group of patients having the same genetic alteration).

The treatment of an individual with a particular therapeutic can be monitored by determining protein e.g., mGluR8 or mGluR8 receptor antagonist and agonist, mRNA and/or transcriptional level. Depending on the level detected, the therapeutic regimen can then be maintained or adjusted (increased or decreased in dose). In a preferred embodiment, the effectiveness of treating a subject with an agent comprises the steps of: (i) obtaining a preadministration sample from a subject prior to administration of the agent; (ii) detecting the level or amount of a protein, mRNA or genomic DNA in the preadministration sample; (iii) obtaining one or more post-administration samples from the subject; (iv) detecting the level of expression or activity of the protein, mRNA or genomic DNA in the post-administration sample; (v) comparing the level of expression or activity of the protein, mRNA or genomic DNA in the preadministration sample with the corresponding protein, mRNA or genomic DNA in the postadministration sample, respectively; and (vi) altering the administration of the agent to the subject accordingly.

Cells of a subject may also be obtained before and after administration of a therapeutic to detect the level of expression of genes other than an mGluR8 gene to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with the therapeutic.

In addition, the ability to target populations expected to show the highest clinical benefit, based on genetic profile can enable: 1) the repositioning of already marketed drugs; 2) the rescue of drug candidates whose clinical development has been discontinued as a result of safety or efficacy limitations, which are patient subgroup-specific; and 3) an accelerated and less costly development for candidate therapeutics and more optimal drug labeling (e.g. since measuring the effect of various doses of an agent on the causative mutation is useful for optimizing effective dose).

In cases in which a SNP leads to a polymorphic protein that is ascribed to be the cause of a pathological condition, method of treating such a condition includes administering to a subject experiencing the pathology the wild type cognate of the polymorphic protein. Once administered in an effective dosing regimen, the wild type cognate provides complementation or remediation of the defect due to the polymorphic protein. The subject's condition is ameliorated by this protein therapy. A subject suspected of suffering from a pathology ascribable to a polymorphic protein that arises from a SNP is to be diagnosed using any of a variety of diagnostic methods capable of identifying the presence of the SNP in the nucleic acid, or of the cognate polymorphic protein, in a suitable clinical sample taken from the subject. Once the presence of the SNP such as any one of the herein disclosed SNPs has been ascertained, and the pathology is correctable by administering a normal or wild-type gene, the subject is treated with a pharmaceutical composition that includes a nucleic acid that harbors the correcting wild-type gene, or a fragment containing a correcting sequence of the wild-type gene.

Non-limiting examples of ways in which such a nucleic acid may be administered include incorporating the wild-type gene in a viral vector, such as an adenovirus or adeno associated virus, and administration of a naked DNA in a pharmaceutical composition that promotes intracellular uptake of the administered nucleic acid. Once the nucleic acid that includes the gene coding for the wild-type allele of the polymorphism is incorporated within a cell of the subject, it will initiate de novo biosynthesis of the wild-type gene product. If the nucleic acid is further incorporated into the genome of the subject, the treatment will have long-term effects, providing de novo synthesis of the wild-type protein for a prolonged duration. The synthesis of the wild-type protein in the cells of the subject will contribute to a therapeutic enhancement of the clinical condition of the subject.

A subject suffering from a pathology ascribed to any one of the novel SNPs disclosed herein may be treated so as to correct the genetic defect. (See Ken et al., Proc. Natl. Acad. Sci. USA 96:10349-10354 (1999)). Such a subject is identified by any method that can detect the polymorphism in a sample drawn from the subject. Such a genetic defect may be permanently corrected by administering to such a subject a nucleic acid fragment incorporating a repair sequence that supplies the wild-type nucleotide at the position of the SNP. This site-specific repair sequence encompasses an RNA/DNA oligonucleotide which operates to promote endogenous repair of a subject's genomic DNA. Upon administration in an appropriate vehicle, such as a complex with polyethylenimine or encapsulated in anionic liposomes, a genetic defect leading to an inborn pathology may be overcome, as the chimeric oligonucleotides induces incorporation of the wild-type sequence into the subject's genome. Upon incorporation, the wild-type gene product is expressed, and the replacement is propagated, thereby engendering a permanent repair.

VI. Therapeutics For Diseases and Conditions Associated with mGluR8 Polymorphisms

Therapeutic for diseases or conditions associated with an mGluR8 polymorphism or haplotype refers to any agent or therapeutic regimen (including pharmaceuticals, nutraceuticals and surgical means) that prevents or postpones the development of or alleviates the symptoms of the particular disease or condition in the subject. The therapeutic can be a polypeptide, peptidomimetic, nucleic acid or other inorganic or organic molecule, preferably a “small molecule” including vitamins, minerals and other nutrients. Preferably the therapeutic can modulate at least one activity of an mGluR8 polypeptide, e.g., interaction with a receptor, by mimicking or potentiating (agonizing) or inhibiting (antagonizing) the effects of a naturally-occurring polypeptide. An agonist can be a wild-type protein or derivative thereof having at least one bioactivity of the wild-type, e.g., receptor binding activity. An agonist can also be a compound that upregulates expression of a gene or which increases at least one bioactivity of a protein. An agonist can also be a compound which increases the interaction of a polypeptide with another molecule, e.g., a receptor. An antagonist can be a compound which inhibits or decreases the interaction between a protein and another molecule, e.g., a receptor or an agent that blocks signal transduction or post-translation processing. Accordingly, a preferred antagonist is a compound which inhibits or decreases binding to a receptor and thereby blocks subsequent activation of the receptor. An antagonist can also be a compound that downregulates expression of a gene or which reduces the amount of a protein present. The antagonist can be a dominant negative form of a polypeptide, e.g., a form of a polypeptide which is capable of interacting with a target peptide, e.g., a receptor, but which does not promote the activation of the receptor. The antagonist can also be a nucleic acid encoding a dominant negative form of a polypeptide, an antisense nucleic acid, or a ribozyme capable of interacting specifically with an RNA. Yet other antagonists are molecules which bind to a polypeptide and inhibit its action. Such molecules include peptides, e.g., forms of target peptides which do not have biological activity, and which inhibit binding to receptors. Thus, such peptides will bind to the active site of a protein and prevent it from interacting with target peptides. Yet other antagonists include antibodies that specifically interact with an epitope of a molecule, such that binding interferes with the biological function of the polypeptide. In yet another preferred embodiment, the antagonist is a small molecule, such as a molecule capable of inhibiting the interaction between a polypeptide and a target receptor. Alternatively, the small molecule can function as an antagonist by interacting with sites other than the receptor binding site.

Modulators of mGlur8 (e.g. mGluR8 receptor antagonist) or a protein encoded by a gene that is in linkage disequilibrium with an mGluR8 gene can comprise any type of compound, including a protein, peptide, peptidomimetic, small molecule, or nucleic acid. Preferred antagonists, which can be identified, for example, using the assays described herein, include nucleic acids (e.g. single (antisense) or double stranded (triplex) DNA or PNA and ribozymes), protein (e.g. antibodies) and small molecules that act to suppress or inhibit mGluR8 transcription and/or protein activity.

VII. Assays to Identify Therapeutics

Based on the identification of mutations that cause or contribute to the development of a disease or disorder that is associated with an mGluR8 polymorphism or haplotype, the invention further features cell-based or cell free assays for identifying therapeutics.

In one embodiment, a cell expressing an mGluR8 receptor on the outer surface of its cellular membrane is incubated in the presence of a test compound alone or in the presence of a test compound and another protein (e.g., a binding partner for the mGluR8 receptor protein/ligand) and the interaction between the test compound and the receptor or between the protein (preferably a tagged protein) and the receptor is detected, e.g., by using a microphysiometer (McConnell et al. (1992) Science 257:1906). An interaction between the receptor and either the test compound or the protein is detected by the microphysiometer as a change in the acidification of the medium. This assay system thus provides a means of identifying molecular antagonists which, for example, function by interfering with protein-receptor interactions, as well as molecular agonist which, for example, function by activating a receptor.

Cellular or cell-free assays can also be used to identify compounds which modulate expression of a mGluR8 gene or a gene in linkage disequilibrium therewith, modulate translation of an mRNA, or which modulate the stability of an mRNA or protein. Accordingly, in one embodiment, a cell which is capable of producing an mGluR8, or other protein is incubated with a test compound and the amount of protein produced in the cell medium is measured and compared to that produced from a cell which has not been contacted with the test compound. The specificity of the compound vis a vis the protein can be confirmed by various control analysis, e.g., measuring the expression of one or more control genes. In particular, this assay can be used to determine the efficacy of antisense, ribozyme and triplex compounds.

An exemplary screening assay of the present invention includes the steps of contacting a variant mGluR8 protein or functional fragment thereof with a test compound or library of test compounds and detecting the formation of complexes. For detection purposes, the molecule can be labeled with a specific marker and the test compound or library of test compounds labeled with a different marker. Interaction of a test compound with the variant protein or fragment thereof can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the variant mGluR8 protein(s) of the invention or functional fragment thereof is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) a variant mGluR8 receptor protein, (ii) an appropriate binding partner thereto, and (iii) a test compound; and (b) detecting interaction of the variant protein and binding partner. A statistically significant change (potentiation or inhibition) in the interaction of the variant protein and the binding partner in the presence of the test compound, relative to the interaction in the absence of the test compound, indicates a potential antagonist (inhibitor). The compounds of this assay can be contacted simultaneously.

Alternatively, a variant mGluR8 receptor protein can first be contacted with a test compound for an appropriate amount of time, following which the binding partner having specificity for the variant receptor protein is added to the reaction mixture. The efficacy of the compound can be assessed by generating dose response curves from data obtained using various concentrations of the test compound. Moreover, a control assay can also be performed to provide a baseline for comparison.

Complex formation between a mGluR8 variant protein of the invention and its binding partner may be detected by a variety of techniques. Modulation of the formation of complexes can be quantitated using, for example, detectably labeled proteins such as radiolabeled, fluorescently labeled, or enzymatically labeled proteins or receptors, by immunoassay, or by chromatographic detection.

VIII. Kits

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting any one or more of the novel polymorphisms disclosed herein. Optional additional components of the kit include, for example, restriction enzymes, reverse-transcriptase or polymerase, the substrate nucleoside triphosphates, means used to label (for example, an avidinenzyme conjugate and enzyme substrate and chromogen if the label is biotin), and the appropriate buffers for reverse transcription, PCR, or hybridization reactions. Usually, the kit also contains instructions for carrying out the methods.

### EXAMPLE 1

GRM8 SNP Patent Methods

Delineation of GRM8 Reference Sequence

The GRM8 locus resides in a region that has been sequenced, assembled and deposited in GenBank database, accession number NT 007933. An 829,973 nucleotide sequence that contains all of the GRM8 exons and corresponds to nucleotides 1,292,101 to 2,122,073 of the Feb. 9, 2001 version of NT 007933 was used as a reference sequence. The reference sequence was utilized to position exons, design primers for amplification of exons by PCR. The position of polymorphisms within the reference sequence NT 007933.7 of 3,761,063 nucleotides updated Dec. 10, 2001 is presented in Table 1.

PCR Conditions:

DNA products containing GRM8 exons were amplified using 1.25 units of TagGold polymerase (Perkin Elmer), in a reaction containing 0.25 uM each dNTP, 1.5 mM MgCl₂ and 1× concentration of Taq polymerase buffer (Perkin Elmer). The reactions were performed in a volume of 0.05 ml with 10 pmole of forward and reverse primers being utilized to amplify the product from 50 ng of human genomic DNA. The PCR primers were designed using the GeneWorks software package on the genomic sequence of GRM8. Human genomic DNA samples from 50 different individuals, representing diversity in the human population, were obtained from the Coriell Cell Repository. Products were amplified with the following PCR cycling conditions:
1 cycle of 10 min at 95° C., 35 cycles of 25 sec at 95° C., 25 sec at 60° C., and 45 sec at 68° C., followed by 1 cycle of 72° C. for 7 min. The various primers are listed in Table 4.
These PCR conditions and primer pair M8×1_{—}1f 5′-ATTGCAATACCACCTGTGG-3′ and M8×1_{—}1r 5′-AACCTGCAGTAGGAGTCATAGC-3′ were used to amplify a 650 base pair product from human genomic DNA containing exon1 of metabotropic glutamate receptor 8. Primer pair M8×2_{—}1f 5′-GTCATGGGTTGAAATGACCC-3′ and M8×2_{—}1r 5′-AGCACTTGGAGATGCTCAGG-3′ were used to amplify a 328 base pair product.
Primer pair 8×3_{—}1f 5′-TGCTCTTAATAGGTGAGAGTGACAC-3′ and M8×3_{—}1r 5′-AGGCAGTCTGTTATTGGAAGG-3′ were used to amplify a 392 base pair product containing exon3.
Primer pair M8×4_{—}1f 5′-TCGGGCAGTTAGAATGATCG-3′ and M8×4_{—}4r 5′-GACAATTCTGCCACCAAAGC-3′ were used to amplify a 434 base pair product containing exon 4.
Primer pair M8×5_{—}1f 5′-GTCCATTCGAAAGTTCTGACA-3′ and M8×5_{—}1r 5′-CCACAGGAAACATTTGAGTGG-3′ were is used to amplify a 235 base pair product containing exon 5.
Primer pair M8×6_{—}1f 5′-GGAAATCTTAGCTCTAATGCTGTC-3′ and M8×6_{—}1r 5′-TTCCACTCTGCCTGGGTATC-3′ were used to amplify a 320 base pair product containing exon 6.
Primer pair M8×7_{—}1f 5′-GGATTGCAATCTTTGCATCAC-3′ and M8×7_{—}1r 5′-AAAGCATCCCTCCTGGAGAG3′ were used to amplify a 321 base pair product containing exon 7.
Exon 8 is a large exon and thus required two primer sets to obtain the entire exon; primer pair M8×8_{—}1f 5′-AACCCGTGGCTAGGATTAGG-3′ and M8×8_{—}1r 5′-GTCGGAAGGAGCATATGATTG-3′ were used to amplify a 532 base pair product and primer pair M8×8_{—}2f 5′-GATTGCAGCACCAGATACAATC-3′ and M8×8_{—}2r 5′-GCACAGACTGAAGCATCTTTAGAG-3′ were used to amplify a 631 base pair product.
Primer pair M8×9_s1f 5′-TTCCCTCAGATGTACATCCAGAC-3′ and M8×9_{—}1r 5′-CTATTAGGAAGTGCTCCCGC-3′ were used to amplify a 310 base pair product containing exon 9.
Primer pair M8×10_{—}1f 5′-GTCGTTGTGCTGTGCATGAC-3′ and M8×10_{—}1r 5′-AAACGGGTTTCTTCACT-3′ were used to amplify a 404 base pair product containing exon 10.

These PCR products were purified using the Qiaquick PCR purification protocol following the manufacturer's instructions for the 96-well format using a vacuum manifold. The PCR product was eluted with 0.06 ml elution buffer and a 0.005 ml aliquot is examined on a 1.5 or 2% agarose gel by standard electrophoresis conditions. The DNA product was visualized on a ultraviolet light box and analyzed to determine the purity of the product and to verify that it is of the expected size.

For each primer set described above the appropriate product was generated from most, if not all, of the genomic DNA samples.

DNA Sequence Analysis

Standard cycle sequencing using approximately 50 ng of the purified PCR product is performed using the BigDye Terminator kit (Applied Biosystems) with the following cycling conditions; 1 cycle 94° C. for 5 min, 24 cycles of 25 sec at 94° C., 25 sec at 50° C., and 4 min at 60° C. The product was purified using a 96-well gel filtration kit (Edge Biosystems) and dried. DNA sequence of the products was determined with either an ABI 377 slab gel system or an ABI 3100 Genetic Analyzer (PE Biosystems). The analyzed DNA sequence files were assembled using the Sequencer software program (PE Biosystems). The assembled sequences were manually inspected for the presence of polymorphisms.

RFLP Analysis of PS6

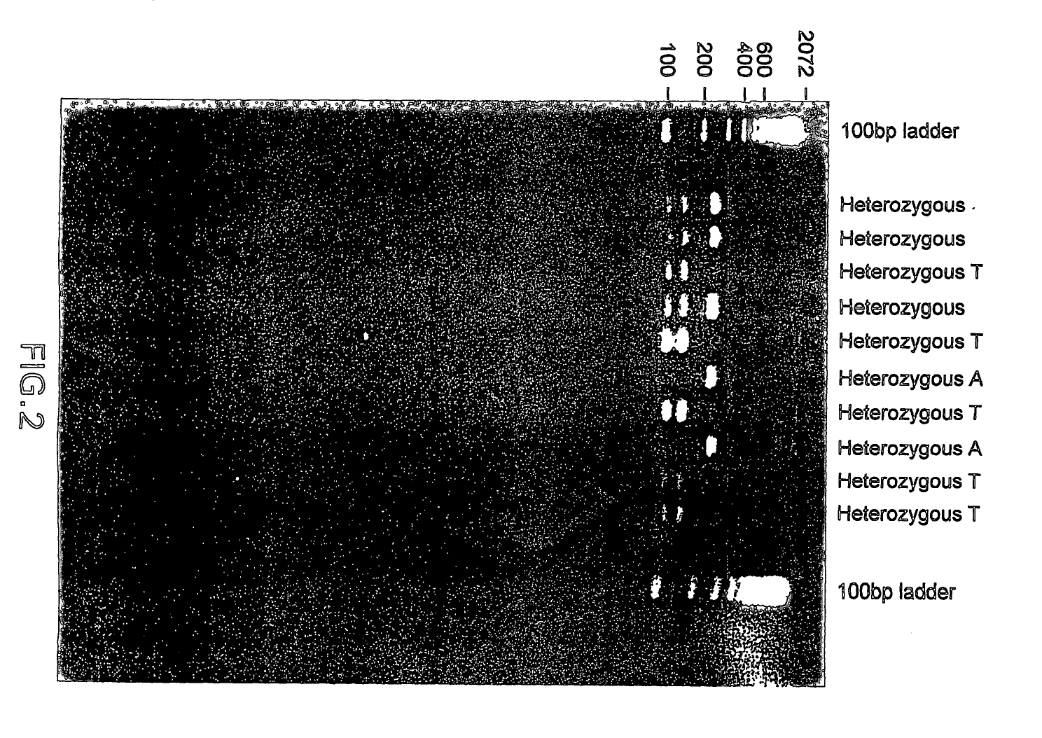

The SNP PS6 located at 1,734,199 in NT07933.6 alters an Eco RI restriction site (GAATTC>GAATAC), this polymorphism is predicted to alter the sequence of the mRNA transcript and of the protein encoded by this transcript, Phe 362 Tyr (TTC>TAC) at position 1095 in XM_{—}045464 and thus is of particular interest. PCR with the primers
M8×5_{—}1f, GTCCATTCGAAAGTTCTGACA; and
M8×5_{—}1r, CCACAGGAAACATTTGAGTGG with the cycling conditions described above was used to generate the fragment corresponding to exon 5. This fragment was then digested with Eco RI and the resulting fragments separated on a 2.5% NuSieve agarose gel (FMC Bioproducts, Rockland Me.) is shown in FIG. 2. Individuals homozygous for the most common allele, the T allele, have the Eco RI site on both chromosomes which results in the band of 235 bp being digested into two fragments of 135 bp and 100 bp. Individuals homozygous for the A allele, will lack the Eco RI site and therefore the PCR product of 235 bp will not be digested by Eco RI. Heterozygous individuals show a mixed pattern, these expected patterns are all observed on FIG. 2 from individuals for which the DNA sequence was determined.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

TABLE 1
Polymorphism Position Relative to NT_007933.7 and Properties

| | SNP position | Change | Frequency | Location | aa Change |
|---|---|---|---|---|---|
| PS1 | 1,392,239 | C/T | 4% | Exon | Phe 119 Silent |
| PS2 | 1,528,555 | T/C | 3% | Exon | Gly 231 Silent |
| PS3 | 1,730,468 | T/C | <1.5% | Exon | lle 265 Thr |
| PS4 | 1,730,897 | A/G | 4% | Intron | NA |
| PS5 | 1,731,127 | A/G | 78% | Intron | NA |
| PS6 | 1,732,472 | T/A | 11% | Exon | Phe 362 Tyr |
| PS7 | 1,865,017 | C/A | 10% | Intron | NA |
| PS8 | 2,101,189 | T/C | <1.4% | Intron | NA |
| PS9 | 2,101,237 | C/G | <1.4% | Exon | Pro 512 Ala |
| PS10 | 2,195,995 | T/C | 43% | Exon | Untranslated |
| | | | | | region |

TABLE 2
GRM8 exon location in NT_007933.7
GRM8 reference sequence: 1-7, 106,047 bp

| Exon | Begin | End |
|---|---|---|
| 1 | 1,391,831 | 1,392,392 |
| 2 | 1,528,373 | 1,528,589 |
| 3 | 1,730,402 | 1,730,537 |
| 4 | 1,730,959 | 1,731,113 |
| 5 | 1,732,406 | 1,732,543 |
| 6 | 1,865,020 | 1,865,220 |
| 7 | 2,025,587 | 2,025,732 |
| 8 | 2,101,198 | 2,102,133 |
| 9 | 2,188,713 | 2,188,959 |
| 10 | 2,195,917 | 2,196,483 |

TABLE 3
Base changes relative to XM_045464

| | Base | Codon | Residue | Change |
|---|---|---|---|---|
| PS1 | 357 | TTC/T | Phe 119 | None |
| PS2 | 693 | GGT/C | Gly 231 | None |
| PS3 | 794 | AT/CT | lle 265 | Thr |
| PS6 | 1095 | TT/AC | Phe 362 | Tyr |
| PS9 | 1534 | C/GCG | Pro 512 | Ala |

TABLE 4

GRM8 Exon Primers
Exon 1: 605 nucleotide product
M8×1_1f
GATTGCAATACCACCTGTGG
M8×1_1r
AACCTGCAGTAGGAGTCATAGC
Exon 2: 328 nucleotide product
M8×2_1f (140)
GTCATGGGTTGAAATGACCC
M8×2_1r (467)
AGCACTTGGAGATGCTCAGG
Exon 3: 392 nucleotide product
8×3_1f
TGCTCTTAATAGGTGAGAGTGACAC
M8×3_1r
AGGCAGTCTGTTATTGGAAGG
Exon 4: 434 nucleotide product
M8×4_1f
TCGGGCAGTTAGAATGATCG
M8×4_4r
GACAATTCTGCCACCAAAGC
Exon 5: 235 nucleotide product
M8×5_1f
GTCCATTCGAAAGTTCTGACA
M8×5_1r
CCACAGGAAACATTTGAGTGG
Exon 6: 320 nucleotide product
M8×6_1f
GGAAATCTTAGCTCTAATGCTGTC
M8×6_1r
TTCCACTCTGCCTGGGTATC
Exon 7: 321 nucleotide product
M8×7_1f (62)
GGATTGCAATCTTTGCATCAC
M8×7_1r (382)
AAAGCATCCCTCCTGGAGAG
Exon 8: 532 nucleotide product
M8×8_1f
AACCCGTGGCTAGGATTAGG
M8×8_1r
GTCGGAAGGAGCATATGATTG
Exon 8: 631 nucleotide product
M8×8_2f
GATTGCAGCACCAGATACAATC
M8×8_2r
GCACAGACTGAAGCATCTTTAGAG
Exon 9: 310 nucleotide product
M8×9_s1f (156)
TTCCCTCAGATGTACATCCAGAC
M8×9_1r (465)
CTATTAGGAAGTGCTCCCGC
Exon 10: 404 nucleotide product
M8×10_1f (54)
GTCGTTGTGCTGTGCATGAC
M8×10_1r (457)
AAACGGGTTTCTTCACT

TABLE 5
GRM8 SNP context

PS1
CTCGACACGTGCTCTAGGGACACCTATGCTTTGGAGCAGTCTCTAACATT
C/T
GTGCAGGCATTAATAGAGAAAGATGCTTCGGATGTGAAGTGTGCTAATGG
PS2
TGGAATTATGTTTCGACACTGGCTTCTGAGGGGAACTATGGTGAGAGCGG
T/C
GTGGAGGCCTTCACCCAGATCTCGAGGGAGATTGGTAAGCATATATTTAT
PS3
TCAGTCACAGAAAATCCCACGTGAACCAAGACCTGGAGAATTTGAAAAAA
T/C
TATCAAACGCCTGCTAGAAACACCTAATGCTCGAGCAGTGATTATGTTTG
PS4
TTAAACAGTGACCTACTGAGTGTATACAACTTCCTAAATCTGGTCTTGTA
A/G
TATTCATAATTGTGGTATTTTTAATACATGTGATATGCATTATTTATTTT
PS5
GCTGTGACAATTTTGCCCAAACGAGCATCAATTGATGGTAAGAATGCACC
A/G
TAGAGAATTTGTTTTATTCCAGTTGGATCTGAACTCAAAGGCAAAACTGG
PS6
TAGAAGCCGAACTCTTGCCAATAATCGAAGAAATGTGTGGTTTGCAGAAT
T/A
CTGGGAGGAGAATTTTGGCTGCAAGTTAGGATCACATGGGAAAAGGAACA
PS7
TCTTTTATTGGAATCTAACATCAACTGATGGTTTTTACTTTTTTATTTTG
C/A
AGGGCTGGAGCGAATTGCTCGGGATTCATCTTATGAACAGGAAGGAAAGG
PS8
GATTAGGATATTATAAATGGGGGAAAAATGGAAGGCTCATTAATTTTTTA
T/C
ACCCACAGGTGGAAGACATGCAGTGGGCTCATAGAGAACATACTCACCCG
PS9
TATACCCACAGGTGGAAGACATGCAGTGGGCTCATAGAGAACATACTCAC
C/G
CGGCGTCTGTCTGCAGCCTGCCGTGTAAGCCAGGGGAGAGGAAGAAAACG
PS10
TTACAGCAATCATTCAATCTGAAACAGGGAAATGGCACAATCTGAAGAGA
T/C
GTGGTATATGATCTTAAATGATGAACATGAGACCGCAAAAATTCACTCCT

1. A nucleic acid molecule comprising a nucleotide sequence selected from the group consisting of:
(a) a nucleotide sequence which is a polymorphic variant of a reference sequence for human mGluR8 gene or a fragment thereof, wherein the reference sequence comprises SEQ ID NO: 1, and the polymorphic variant comprises at least one polymorphism selected from the group consisting of T at position 1,392,239 (C/T, C at position 1,528,555 (T/C), C at position 1,730,468 (T/C), G at position 1,730,897 (A/G), G at position 1,731,127 (A/G), A at position 1,732,472 (T/A), A at position 1,865,017(C/A), C at position 2,101,189 (T/C), G at position 2,101,237 (C/G), and C at position 2,195,995 (T/C) as defined by the position in NT007933.7 updated Dec. 10, 2001:
(b) a complementary nucleotide sequence comprising a sequence complementary to one or more of said polymorphic sequences set forth in (a) above:
(c) an antisense sequence thereto: and
(d) a fragment thereof of at least 20 bases comprising at least one polymorphic site is said fragment wherein said single nucleotide polymorphism is associated with a genetic predisposition for a disease selected from the group consisting of schizophrenia, Parkinson's disease, Alzheimer's disease, Huntington's disease, stroke, anxiety, cognitive dysfunction, attention deficit hyperactivity disorder, autism, pain and inflammation.

2. The nucleic acid molecule of claim 1, wherein said nucleic acid is genomic DNA, cDNA, or mRNA.

3. (canceled)

4. The nucleic acid molecule of claim 1, wherein said nucleic acid molecule contains at least one single nucleotide polymorphism at position 1,392,239.

5. The nucleic acid molecule of claim 1, wherein said nucleic acid molecule contains at least one single nucleotide polymorphism at position 1,528,555.

6. The nucleic acid molecule of claim 1, wherein said nucleic acid molecule contains at least one single nucleotide polymorphism at position 1,730,468.

7. The nucleic acid molecule of claim 1, wherein said nucleic acid molecule contains at least one single nucleotide polymorphism at position 1,730,897.

8. The nucleic acid molecule of claim 1, wherein said nucleic acid molecule contains at least one single nucleotide polymorphism at position 1,731,127.

9. The nucleic acid molecule according to claim 1, further comprising a detectable label.

10. (canceled)

11. (canceled)

12. (canceled)

13. An expression vector comprising the nucleic acid molecule of claim 1.

14. A recombinant non-human organism transfected with the nucleic acid molecule of claim 1, wherein the organism expresses an mGluR8 receptor protein encoded by the nucleotide sequence.

15. (canceled)

16. An allele-specific primer capable of detecting a mGluR8 gene polymorphism wherein said polymorphism is located at a position corresponding to position 1,392,239 on exon 1; at position 1,528,555 on exon 2; at position 1,730,468 on exon 3; at position 1,732,472 on exon 5; at position 2,101,237 on exon 8; at position 2,195,995 on exon 10; and/or at position 1,730,897 on intron 3; at position 1,731,127 on intron 4; at position 1,865,017 on intron 5; or at position 2,101,189 on intron 8; as defined by the position in NT 007933.7.

17. A nucleic acid molecule comprising a nucleotide sequence which is a polymorphic variant of a reference sequence for the human mGluR8 cDNA, wherein the reference sequence comprises SEQ ID NO:2 as defined by the position in ACCESSION NO. XM045464 and the polymorphic variant comprises at least one polymorphism selected from the group consisting of thymine (T) at a position corresponding to nucleotide 357; cytosine (C) at a position corresponding to nucleotide 693; cytosine (C) at a position corresponding to nucleotide 794; adenine (A) at a position corresponding to nucleotide 1095 and guanine (G) at a position corresponding to nucleotide 1534; or a complementary strand thereof or an antisense sequence thereto or a fragment thereof of at least 20 bases comprising at least one polymorphism.

18. A host cell transformed or transfected with the nucleic acid molecule of claim 17, under conditions favoring expression of a mGluR8 receptor protein encoded by the polymorphic variant sequence.

19. A polypeptide comprising an amino acid sequence which is a polymorphic variant of a reference sequence for the human mGluR8 receptor protein or a fragment thereof, wherein the reference sequence comprises SEQ ID NO:3 and the polymorphic variant comprises at least one polymorphism selected from the group consisting of threonine at a position corresponding to amino acid position 265, tyrosine at a position corresponding to amino acid position 362 and alanine at a position corresponding to amino acid position 512.

20. An isolated antibody specific for and immunoreactive with the polypeptide of claim 19.

21-23. (canceled)

24. A method of detecting a polymorphic site in a nucleic acid molecule, the method comprising:
(a) contacting said nucleic acid molecule with an oligonucleotide probe under conditions favoring hybridization between the probe and the nucleic acid molecule, wherein said probe comprises the nucleic acid molecule of claim 11; and
(b) determining binding between said nucleic acid molecule and said oligonucleotide probe to form a complex, wherein presence of said complex indicates the presence of a polymorphic site in said nucleic acid.

25. A method for the diagnosis of a single nucleotide polymorphism in a sample nucleic acid molecule suspected of containing a single nucleotide polymorphism in a mGluR8 gene, which method comprises detecting in said gene the presence or absence of one or more single nucleotide polymorphism defined by the presence of thymine (T) at position 1,392,239; cytosine (C) at position 1,528,555; cytosine (C) at position 1,730,468; guanine (G) at position 1,730,897; guanine (G) at position 1,731,127; adenine (A) at position 1,732,472; adenine (A) at position 1,865,017; cytosine (C) at position 2,101,189; guanine (G) at position 2,101,237; cytosine (C) at position 2,195,995 in SEQ ID NO:1, wherein the presence of at least one variant mGluR8 allele in said sample nucleic acid is taken as an indication of the presence of a single nucleotide polymorphism in said sample.

26. A method of treating a human in need of treatment with a mGluR8 receptor antagonist drug in which the method comprises:
(i) diagnosis of a single nucleotide polymorphism in the mGluR8 gene in the human, which diagnosis comprises determining the sequence of the nucleic acid at a position corresponding to at least one nucleotide at position 1,392,239; 1,528,555; 1,730,468; 1,730,897; 1,731,127; 1,732,472; 1,865,017; 2,101,189; 2,101,237 and 2,195,995 relative to SEQ ID NO:1, wherein said single nucleotide polymorphism at position 1,392,239 is presence of T; the single nucleotide polymorphism at position 1,528,555 is presence of C; the single nucleotide polymorphism at position 1,730,468 is presence of C; the single nucleotide polymorphism position 1,730,897 is presence of G; the single nucleotide polymorphism at position 1,731,127 is presence of G; the single nucleotide polymorphism at position 1,732,472 is presence of A; the single nucleotide polymorphism at position 1,865,017 is presence of A; the single nucleotide polymorphism at position 2,101,189 is presence of C; and the single nucleotide polymorphism at position 2,101,237 is presence of G, and the single nucleotide polymorphism at position 2,195,995 is presence of C; said positions being relative to SEQ ID NO: 1; and
(ii) administering an effective amount of a mGluR8 receptor antagonist drug.

27. A method for screening for drugs targeting the isolated polypeptide of claim 19 which comprises contacting the polymorphic variant with a candidate agent and assaying for binding activity.

28. A method for identifying an association between a trait and at least one genotype or haplotype of a mGluR8 gene which comprises comparing the frequency of the genotype or haplotype in a population exhibiting the trait with the frequency of the genotype or haplotype in a reference population, wherein the genotype or haplotype comprises a nucleotide pair or nucleotide located at one or more polymorphic sites selected from the group consisting of thymine (T) at position 1,392,239; cytosine (C) at position 1,528,555; cytosine (C) at position 1,730,468; guanine (G) at position 1,730,897; guanine (G) at position 1,731,127; adenine (A) at position 1,732,472; adenine (A) at position 1,865,017; cytosine (C) at position 2,101,189; guanine (G) at position 2,101,237; cytosine (C) at position 2,197, 722 said positions being relative to SEQ ID NO: 1, wherein a higher frequency of the genotype or haplotype in the trait population than in the reference population indicates the trait is associated with the genotype or haplotype.

29. A method for diagnosing a genetic predisposition for a disease, condition or disorder in a subject comprising, obtaining a biological sample containing nucleic acid from said subject; and analyzing said nucleic-acid to detect the presence or absence of a single nucleotide polymorphism in SEQ ID NO: 1 or the complement thereof, wherein said single nucleotide polymorphism is associated with a genetic predisposition for a disease selected from the group consisting of schizophrenia, Parkinson's disease, Alzheimer's disease, Huntington's disease, stroke, anxiety, cognitive dysfunction, attention deficit hyperactivity disorder, autism, pain and inflammation.

30-31. (canceled)

32. A method for diagnosing a genetic predisposition for a disease, condition or disorder in a subject comprising, obtaining a biological sample containing nucleic acid from said subject; and analyzing said nucleic-acid to detect the presence or absence of a single nucleotide polymorphism in SEQ ID NO:2 or the complement thereof, wherein said single nucleotide polymorphism is associated with a genetic predisposition for a disease selected from the group consisting of schizophrenia, Parkinson's disease, Alzheimer's disease, Huntington's disease, stroke, anxiety, cognitive dysfunction, attention deficit hyperactivity disorder, autism, pain and inflammation.

33-35. (canceled)

36. A computer-readable storage medium for storing data for access by an application program being executed on a data processing system, comprising:
(i) a data structure stored in the computer-readable storage medium, the data structure including information resident in a database used by the application program and including: and
(ii) a plurality of records, each record of the plurality comprising information identifying a polymorphisms as claimed in claim 24.

37. The computer-readable storage medium of claim 36, wherein each record has a field identifying a base occupying a polymorphic site and a location of the polymorphic site.

38. (canceled)

39. A method of screening human subjects for susceptibility to Parkinson's disease, which method comprises screening for the presence or absence in the genome of the human subject of one or more variant mGluR8 alleles selected from the group consisting of mGluR8 exon 1 1,392,239 T, mGluR8 exon 2 1530,282 C, mGluR8 exon 3 1,730,468 C, mGluR8 intron 3 1,730,897 G; mGluR8 intron 4 1,731,127 A, mGluR8 exon 5 1,732,472 A, mGluR8 intron 5 1,865,017 A; mGluR8 intron 8 2,101,189 C, mGluR8 exon 8 2,101,237 G, and mGluR8 exon 10 2,195,995 C, wherein the presence of at least one variant mGluR8 allele in the genome of said human subject is taken as an indication of susceptibility to Parkinson's disease.

40-41. (canceled)

42. A method for determining the effectiveness of treating a subject that has or is predisposed to developing a disease or condition that is associated with an human mGluR8 allelic pattern, comprising at least one allelic variation at a position corresponding with a particular dose of a particular therapeutic, comprising the steps of:
a) detecting the level, amount or activity of an human mGluR8 protein or an human mGluR8 mRNA in a sample obtained from a subject;
b) administering the particular dose of the particular therapeutic to the subject and detecting the level, amount of activity of an human mGluR8 protein or an human mGluR8 mRNA in a sample obtained from a subject; and
c) comparing the relative level, amount or activity obtained in step a) with the level, amount or activity obtained in step b), wherein an increase in the relative amount or activity of the human mGluR8 protein or mRNA after administration of the therapeutic as compared to that before administration of the therapeutic indicates that the particular dose of the particular therapeutic is effective in treating the subject.

43. A method of claim 42, wherein the therapeutic is a modulator of a human mGluR8 activity.

44. A method for screening for a therapeutic human mGluR8 agonist or antagonist for treating or preventing a disease or condition that is associated with a single nucleotide polymorphism in the mGluR8 gene, comprising the steps of
a) combining an human mGluR8 polypeptide or bioactive fragment thereof, an human mGluR8 binding partner and a test compound which is not known to affect a human mGluR8 bioactivity under conditions wherein, but for the test compound, the human mGluR8 protein and human mGluR8 binding partner are able to interact; and
b) detecting the extent to which, in the presence of the test compound, a human mGluR8 protein/binding partner complex is formed, wherein an increase in the amount of said complex in the presence of the compound relative to that in the absence of the compound indicates that the compound is a human mGluR8 agonist therapeutic and a decrease in the amount of complex in the presence of the compound relative to that in the absence of the compound indicates that the compound is an human mGluR8 antagonist therapeutic for treating or preventing the disease or condition.

45. A method for identifying a therapeutic for treating or preventing a disease or condition that is associated with a single nucleotide polymorphism in the mGluR8 gene, comprising the steps of:
a) contacting an appropriate amount of a candidate compound with a cell or cellular extract, which expresses a gene encoding a human mGluR8 receptor protein that provides a human mGluR8 agonist or antagonist protein bioactivity; and
b) determining the resulting human mGluR8 protein bioactivity, wherein a decrease of an human mGluR8 agonist bioactivity or an increase in an human mGluR8 antagonist bioactivity in the presence of the compound as compared to the bioactivity in the absence of the compound indicates that the candidate compound is an effective therapeutic.
